SEQUENCE LISTING (26 sequences)

SEQ ID NO: 1 - RNA, 22 nt, synthetic construct
  source 1..22: mol_type = other RNA
  ncRNA 1..22: ncRNA_class = miRNA
tcgtaccgtgagtaataatgcg

SEQ ID NO: 2 - RNA, 22 nt, synthetic construct
  source 1..22: mol_type = other RNA
  ncRNA 1..22: ncRNA_class = miRNA
cagtgcaatgttaaaagggcat

SEQ ID NO: 3 - RNA, 22 nt, synthetic construct
  source 1..22: mol_type = other RNA
  ncRNA 1..22: ncRNA_class = miRNA
tagcttatcagactgatgttga

SEQ ID NO: 4 - RNA, 22 nt, synthetic construct
  source 1..22: mol_type = other RNA
  ncRNA 1..22: ncRNA_class = miRNA
ttcaagtaatccaggataggct

SEQ ID NO: 5 - RNA, 22 nt, synthetic construct
  source 1..22: mol_type = other RNA
  ncRNA 1..22: ncRNA_class = miRNA
actggacttggagtcagaaggc

SEQ ID NO: 6 - RNA, 22 nt, synthetic construct
  source 1..22: mol_type = other RNA
  ncRNA 1..22: ncRNA_class = miRNA
tgagaactgaattccatgggtt

SEQ ID NO: 7 - RNA, 22 nt, synthetic construct
  source 1..22: mol_type = other RNA
  ncRNA 1..22: ncRNA_class = miRNA
acagtagtctgcacattggtta

SEQ ID NO: 8 - RNA, 21 nt, synthetic construct
  source 1..21: mol_type = other RNA
  ncRNA 1..21: ncRNA_class = miRNA
taattttatgtataagctagt

SEQ ID NO: 9 - RNA, 22 nt, synthetic construct
  source 1..22: mol_type = other RNA
  ncRNA 1..22: ncRNA_class = miRNA
tttggtccccttcaaccagctg

SEQ ID NO: 10 - RNA, 21 nt, synthetic construct
  source 1..21: mol_type = other RNA
  ncRNA 1..21: ncRNA_class = miRNA
cattattacttttggtacgcg

SEQ ID NO: 11 - RNA, 22 nt, synthetic construct
  source 1..22: mol_type = other RNA
  ncRNA 1..22: ncRNA_class = miRNA
gctcttttcacattgtgctact

SEQ ID NO: 12 - RNA, 23 nt, synthetic construct
  source 1..23: mol_type = other RNA
  ncRNA 1..23: ncRNA_class = miRNA
cctcctgactccaggtcctgtgt

SEQ ID NO: 13 - RNA, 23 nt, synthetic construct
  source 1..23: mol_type = other RNA
  ncRNA 1..23: ncRNA_class = miRNA
cccagtgttcagactacctgttc

SEQ ID NO: 14 - RNA, 22 nt, synthetic construct
  source 1..22: mol_type = other RNA
  ncRNA 1..22: ncRNA_class = miRNA
gagcttattcataaaagtgcag

SEQ ID NO: 15 - RNA, 22 nt, synthetic construct
  source 1..22: mol_type = other RNA
  ncRNA 1..22: ncRNA_class = miRNA
agctggtaaaatggaaccaaat

SEQ ID NO: 16 - RNA, 21 nt, synthetic construct
  source 1..21: mol_type = other RNA
  ncRNA 1..21: ncRNA_class = miRNA
caacaccagtcgatgggctgt

SEQ ID NO: 17 - RNA, 22 nt, synthetic construct
  source 1..22: mol_type = other RNA
  ncRNA 1..22: ncRNA_class = miRNA
cctattcttggttacttgcacg

SEQ ID NO: 18 - RNA, 22 nt, synthetic construct
  source 1..22: mol_type = other RNA
  ncRNA 1..22: ncRNA_class = miRNA
cctctgaaattcagttcttcag

SEQ ID NO: 19 - RNA, 22 nt, synthetic construct
  source 1..22: mol_type = other RNA
  ncRNA 1..22: ncRNA_class = miRNA
tgcctactgagctgatatcagt

SEQ ID NO: 20 - RNA, 22 nt, synthetic construct
  source 1..22: mol_type = other RNA
  ncRNA 1..22: ncRNA_class = miRNA
tgcctactgagctgaaacacag

SEQ ID NO: 21 - RNA, 22 nt, synthetic construct
  source 1..22: mol_type = other RNA
  ncRNA 1..22: ncRNA_class = miRNA
tggctcagttcagcaggaacag

SEQ ID NO: 22 - RNA, 22 nt, synthetic construct
  source 1..22: mol_type = other RNA
  ncRNA 1..22: ncRNA_class = miRNA
accgtggctttcgattgttact

SEQ ID NO: 23 - RNA, 22 nt, synthetic construct
  source 1..22: mol_type = other RNA
  ncRNA 1..22: ncRNA_class = miRNA
taacagtctacagccatggtcg

SEQ ID NO: 24 - RNA, 24 nt, synthetic construct
  source 1..24: mol_type = other RNA
  ncRNA 1..24: ncRNA_class = miRNA
gctggtttcatatggtggtttaga

SEQ ID NO: 25 - RNA, 22 nt, synthetic construct
  source 1..22: mol_type = other RNA
  ncRNA 1..22: ncRNA_class = miRNA
ctggtttcacatggtggcttag

SEQ ID NO: 26 - RNA, 23 nt, synthetic construct
  source 1..23: mol_type = other RNA
  ncRNA 1..23: ncRNA_class = miRNA
tagcaccatttgaaatcagtgtt

(19) United States
(12) Patent Application Publication
(10) Pub. No.: US 2026/0306537 A1
(43) Pub. Date: Oct. 8, 2026

(54) INTRANASAL DELIVERY OF CD34+ CELLS FOR THE TREATMENT OF STROKE

(71) Applicant: CELLPROTHERA SAS, Mulhouse (FR)

(72) Inventors: Ibon GARITAONANDIA, Mulhouse (FR); Christine VIGNON, Mulhouse (FR); Matthieu DE KALBERMATTEN, Mulhouse (FR)

(73) Assignee: CELLPROTHERA SAS, Mulhouse (FR)

(21) Appl. No.: 19/477,192

(22) PCT Filed: Apr. 19, 2024
(86) PCT No.: PCT/EP2024/060796

§ 371 Date: Oct. 21, 2025

(30) Foreign Application Priority Data
Apr. 21, 2023 (EP) 23315096.0

(51) Int. Cl. A61K 35/14; A61K 9/00; A61K 35/12; A61K 35/51; A61P 9/10; C12N 5/078

(52) U.S. Cl. CPC A61K 35/14; A61K 9/0043; A61K 35/51; A61P 9/10; C12N 5/0634; A61K 2035/124

(57) ABSTRACT

The disclosure relates to methods of treatment comprising intranasal delivery of CD34+ cells and CD34+ cells for use in such methods.

### CROSS REFERENCE TO RELATED APPLICATION(S)

This application is a 35 U.S.C. § 371 National Stage of International Patent Application No. PCT/EP2024/060796, filed Apr. 19, 2024, claiming benefit from European Patent Application No. 23315096.0, filed Apr. 21, 2023, designating the United States.

### SEQUENCE LISTING STATEMENT

The instant application contains a Sequence Listing in electronic format which has been submitted via EFS-Web. Said Sequence Listing, created on Apr. 19, 2024, is named “P216318WO-Sequence-Listing-ST26.xml” and is 33,151 bytes in size. The information in the electronic format of the Sequence Listing is part of the present application and is incorporated herein by reference in its entirety.

### TECHNICAL FIELD

The disclosure relates to methods of treatment comprising intranasal delivery of CD34+ cells and CD34+ cells for use in such methods.

### BACKGROUND

CD34 is a cell surface marker used to identify and isolate hematopoietic stem/progenitor cells (HSPCs). CD34+ cells can be isolated from blood samples using immunomagnetic techniques. CD34+ cells can differentiate into all types of blood cells as well as endothelial cells.

CD34+ cells can be collected via leukapheresis. Leukapheresis comprises removing blood from the body and then separating the white blood cells before returning the blood to the body. Where leukapheresis is used to collect CD34+ cells, typically a subject is first administered a hematopoietic growth factor. Alternatively, CD34+ cells can be expanded from a whole blood sample. An automated device (StemXpand®) that allows stem cell expansion after granulocyte colony-stimulating factor (G-CSF) mobilisation has been developed and shown to provide CD34+ cell numbers at least equivalent to those collected during leukapheresis (Saucourt, et al. 2019). Characteristics (CD34+ cell numbers, purity/impurity profile, and viability) and safety (sterility, pyrogen, and mycoplasma content) of the expanded cells have been assessed and the functionality of expanded cells (eCD34+) has been demonstrated in an in vivo preclinical study in rats (Saucourt et al., 2019).

There are three different types of strokes: ischaemic stroke, haemorrhagic stroke, and transient ischaemic attack (TIA). Ischaemic stroke is the most common and is caused by a blockage cutting off the blood supply to the brain. Haemorrhagic stroke is caused by bleeding in the brain. TIA is a mini-stroke and has the same symptoms as ischaemic stroke but only lasts for a short amount of time because the blockage of the blood supply is temporary. In ischaemic stroke the blood supply to part of the brain is interrupted, preventing brain tissue from getting oxygen and nutrients and leading to the death of brain cells within minutes. Damage to the brain cells can affect motor and cognitive functions. The effects of an ischaemic stroke depend on where it takes place in the brain and the size of the damaged area. The interruption of the blood supply to the brain usually occurs when a blood clot blocks the flow of blood to the brain or blood flow to the brain is otherwise blocked by a dysfunction in a blood vessel. These blood dots typically form in areas where the arteries have been narrowed or blocked over time by fatty deposits known as plaques. There are two types of ischaemic stroke: thrombotic strokes and embolic strokes. Thrombotic strokes are caused by a thrombus (blood clot) that develops in the arteries supplying blood to the brain and embolic strokes are caused by a blood clot that develops elsewhere in the body and then travels through the bloodstream to the brain.

The use of CD34+ cells in the treatment of ischaemic stroke has been investigated. In these studies, intra-arterial (Banerjee S et al., 2014.), intravenous (Nystedt J et al., 2006), intrathecal (Wang L et al., 2013) and intracerebral (Chen D C et al., 2014) administration routes were investigated. Problems associated with the intra-arterial and intravenous routes of administration are that they lead to systemic exposure and deliver a very small percentage of the cells to the brain because they do not bypass the blood brain barrier. The intrathecal and intracerebral routes of administration deliver the cells directly to the peri-infarct area, maximizing the chances of engraftment around the target lesion site. However, the intrathecal and intracerebral routes are invasive routes and add significant risk to the patient. The intracerebral route in particular requires complicated stereotactic surgery, which carries the risk of intracranial haemorrhage. There is therefore a need for improved treatments for stroke.

### SUMMARY

The Applicant has shown that intranasal administration of CD34+ cells results in the rapid delivery of cells to the brain. Intranasal administration delivers cells to the brain and spinal cord along the olfactory and trigeminal pathways involving perineural and perivascular channels and leads to a disperse biodistribution of cells throughout the central nervous system.

This is advantageous as the intranasal route is non-invasive and significantly reduces the risk to the patient as compared to other routes of administration. Furthermore, the Applicant has shown that intranasal administration of CD34+ cells leads to locomotor and neurological improvements, reduction in infarct size, cell loss, and inflammation, and increase in angiogenesis and neurogenesis in the conte)d of ischemic stroke. Surprisingly, the intranasal administration of CD34+ cells provides the same locomotor and neurological improvements, reduction in infarct size, cell loss, and inflammation, and increase in angiogenesis and neurogenesis as administration of CD34+ cells via the intracerebral route.

The invention therefore provides a population of CD34+ cells for use in a method for treating stroke, the method comprising intranasally administering said population of CD34+ cells to a subject.

Aspects of the invention further provide a population of CD34+ cells for use in a method of:
(a) treating stroke, wherein the CD34+ cells are administered to a subject intranasally, wherein the CD34+ cells secrete paracrine factors in an area affected by stoke thereby treating stroke; and/or
(b) treating stroke, wherein the CD34+ cells are administered to a subject intranasally, wherein the CD34+ cells promote angiogenesis in an area affected by stroke, thereby treating stroke; and/or
(c) treating stroke, wherein the CD34+ cells are administered to a subject intranasally, and wherein the CD34+ cells promote neurogenesis; and/or
(d) stimulating angiogenesis in a subject; and/or
(e) stimulating neurogenesis in a subject; and/or
(f) treating stroke, wherein the CD34+ cells are administered to a subject intranasally, and wherein the CD34+ cells promote reduction in infarct size, and/or reduce cell loss in the infarct area and/or peri-infarct area and/or inflammation in the infarct area and/or peri-infarct area, thereby treating stroke; and/or
(g) treating stroke, wherein the CD34+ cells are administered to a subject intranasally, and wherein the CD34+ cells promote motor and/or neurological recovery, thereby treating stroke.

The CD34+ cells may be isolated from umbilical cord blood or whole blood.

The CD34+ cells may be administered to the subject within 1 day, 2 days, 3 days, or within 4 days, of the stroke event.

The methods disclosed herein may comprise administering at least 7×10⁶ CD34+ cells.

The CD34+ cells may express one or more paracrine factors, and/or VEGF.

The amount of VEGF expressed by the CD34+ cells into a culture medium may be at least about 150 μg/ml, or the amount of VEGF expressed by the CD34+ cells into a culture medium may be at least about 4 fg/cell.

The CD34+ cells may express one or more or each of miR126, miR130a, miR21, miR26a, miR378a, miR146a, miR21, miR199a, miR590, miR133a, miR-24, miR29b, and miR132.

The CD34+ cell viability may be at least about 90%, and/or the CD34+ cell purity may be at least about 75%.

The CD34+ cells may be a cultured and/or expanded population, and/or purified, and/or an isolated population.

The population of CD34+ cells may comprise
(i) at least about 75% CD34+ cells, and/or
(ii) about 15% monocytes or lower, and/or
(iii) about 5% granulocytes or lower, and/or
(iv) about 3% lymphocytes or lower.

The number of cells administered per dose may be about 7×10⁶.

The volume of cells administered per nostril may be about 1 ml and/or the total volume administered per dose may be about 2 ml.

The CD34+ cells may be provided as a sterile suspension, and/or the population may be provided in the form of a pharmaceutical composition, optionally comprising buffer.

The CD34+ cells may increase the expression of doublecortin DCX and/or VEGFR1.

### BRIEF DESCRIPTION OF THE DRAWINGS

FIG. 1A-D: Behavioural studies. Behaviour testing. (a) Motor activity revealed by elevated body swing test (EBST). Middle cerebral artery occlusion (MCAO) rats transplanted with expanded CD34+ cells (ProtheraCytes®) via intra-arterial/intravascular (IV), intracerebral (IC), and intranasal (IN) routes of administration displayed significantly less asymmetry on day 7, 14, 28 than their corresponding vehicle control MCAO animals (****p<0.0001) (Media). (b) Motor activity revealed by Cylinder Test. MCAO rats transplanted with ProtheraCytes® via IV, IC, and IN routes of administration demonstrated significantly more use of impaired forelimb than their corresponding vehicle control MCAO animals (*p<0.05, “p<0.01, ***p<0.001, ****p<0.0001). (c) Motor activity revealed by Grip Strength. MCAO rats transplanted with

ProtheraCytes® via IV, IC, and IN routes of administration presented significantly less impaired paw grasp than their corresponding vehicle control MCAO animals (*p<0.05, “p<0.01, ***p<0.001, ****p<0.0001). (d) Motor activity revealed by Balance Beam. MCAO rats transplanted with ProtheraCytes® via IV, IC, and IN routes of administration showed significantly better motor coordination during beam walks than their corresponding vehicle control MCAO animals (****p<0.0001).

FIG. 2: Histological studies. Quantitative measurement of infarct areas. Nissl staining for coronal brain sections showing infarct areas (black outline). MCAO rats transplanted with ProtheraCytes® via IV, IC, and IN routes of administration displayed significantly smaller infarct areas than the vehicle control MCAO animals (*p<0.05, **p<0.01, ***p<0.001, ****p<0.0001).

FIG. 3: Quantitative analysis of cell survival in the peri-infarct area. MCAO rats transplanted with ProtheraCytes® via IV, IC, and IN routes of administration showed significantly more living cells in the peri-infarct area than vehicle control MCAO animals (*p<0.05, **p<0.01, ***p<0.001, ****p<0.0001). Scale bar=50 μm.

FIG. 4. Quantitative analysis of inflammation and cell engraftment. MCAO rats transplanted with ProtheraCytes® via IV, IC, and IN routes of administration showed a reduction in inflammation (Iba-1+ cells, black arrowheads) compared to vehicle control MCAO rats (***p<0.001). Survival and engraftment of ProtheraCytes® (white arrowheads) via IV, IC, and IN routes of administration was demonstrated in the implanted MCAO rats compared to the vehicle control MCAO rats where no ProtheraCytes® were found (*p<0.05, **p<0.01, ***p<0.001, ****p<0.0001). Scale bar=50 μm.

FIG. 5. Quantitative analysis of neurogenesis and cell engraftment. MCAO rats transplanted with ProtheraCytes® via IV, IC, and IN routes of administration showed significantly higher neurogenesis (doublecortin DCX+ cells, black arrowheads) than vehicle control MCAO animals (*p<0.05, **p<0.01, ***p<0.001, ****p<0.0001). Engraftment of ProtheraCytes® (white arrowheads) was again demonstrated with all three routes of administration (**p<0.01, ***p<0.001, ****p<0.0001). Scale bar=50 μm.

FIG. 6. Quantitative analysis of angiogenesis and cell engraftment. MCAO rats transplanted with ProtheraCytes® via IV, IC, and IN routes of administration had increased angiogenesis (vascular endothelial growth factor receptor 1 VEGFR1+ cells, black arrowheads) compared to vehicle controls MCAO rats (*p<0.05, “p<0.01, ***p<0.001, ****p<0.0001). Engraftment of ProtheraCytes® (white arrowheads) was again demonstrated with ail three routes of administration and increased angiogenesis was observed close to the implanted human cells, supporting the mechanism of action of CD34+ cells, which promote angiogenesis through the secretion of paracrine factors such as VEGF (***p<0.001, ****p<0.0001). Scale bar=50 μm.

FIG. 7. Quantitative analysis of exosome secretion. MCAO rats transplanted with ProtheraCytes® via IV, IC, and IN routes of administration had significantly higher number of extracellular vesicles (tetraspin CD63+, black arrowheads) than vehicle control MCAO rats (***p<0.001, ****p<0.0001). Scale bar=50 μm. This important finding shows that ProtheraCytes® secrete extracellular vesicles in vivo and supports the results obtained in vitro, where we showed that CD34+ cells secrete exosomes with pro-angiogenic and anti-apoptotic miRNAs. It provides further evidence of the mechanism of action of ProtheraCytes® showing that these cells also promote neurogenesis and angiogenesis through the secretion of CD63-positive extracellular vesicles. There were no signs of toxicity or tumors in any of the transplanted animals.

FIG. 8: VEGF as a potency test (A, B, C) Concentration of Vascular Endothelial Growth Factor (VEGF) in cell culture supernatants after 9 days of CD34+ cell expansion from four healthy donors and 16 patients with acute myocardial infarction (EXCELLENT study). No significant difference observed when VEGF concentration was compared between patients and healthy donors, but a significant difference was observed between patients and vehicle control (StemFeed®) (p=0.0007) and healthy donors and StemFeed® (p=0.0087). When the VEGF concentration was normalized by the number of CD34+ cells, we observed that there was no significant difference between the VEGF secreted per cell in healthy donors (4.1 fg/cell) and AMI patients (4.4 fg/cell) (p=0.6343). (D, E) VEGF concentration and CD34+ cells after expansion: significant correlation between VEGF concentration and the number of CD34+ cells after expansion (Pearson correlation coefficient=0.7484, p value=0.0009). (F) In vitro tube formation assay: HUVECs cocultured with ProtheraCytes® supernatant have significantly higher tube formation than HUVECs cocultured with vehicle control (StemFeed®) (t-test, p=0.0082). The pictures display representative brightfield images of tube formation. Scale bar=20 μm.

FIG. 9: ProtheraCytes®-derived exosome characterization (A) The average size of Protheracytes®-derived exosomes was determined to be 86.7±10.17 nm. (B) The number of exosomes secreted by ProtheraCytes® increased over time from around 6000 extracellular vesicles/cell at 30 minutes to 16000 extracellular vesicles/cell at 2 hours. (C) Protheracytes®-derived exosomes express the characteristic exosomal markers CD63, CD81 and CD9 as well as endothelial (CD49e and CD44) and stem cell markers (CD133).

FIG. 10: Proangiogenic, anti-apoptotic, anti-fibrosis and cardiac miRNAs relative expression by ProtheraCytes® and their secreted exosomes. (A) Total RNA from ProtheraCytes® (Cells) and their exosomes (Exosomes) collected from AMI patients (n=7) were subjected to real time PCR and normalized to small RNA (let-7a). miRNA expression in the exosomes secreted from ProtheraCytes® was significantly higher than in ProtheraCytes® (Mann-Whitney test). (B) Comparative fold change expression of exosomes versus cells representation of the indicated miRNA collected from AMI patients (n=7).

### DETAILED DESCRIPTION

### Definitions

CD34+ cells comprise hematopoietic stem/progenitor cells (HSPCs) as well as endothelial progenitor cells (EPCs). HSPCs can differentiate into all blood cell types and EPCs can differentiate into endothelial cells. CD34+ cells can be mobilized from the bone marrow into the peripheral blood by the administration of granulocyte colony stimulating growth factor(G-CSF). CD34+ cells may also be obtained from umbilical cord blood. Total CD34+ cells represent approximately 0.5-1% of total bone marrow derived mononuclear cells. CD34+ cells grow in suspension cultures.

“VEGF” (vascular endothelial growth factor) is a potent proangiogenic growth factor known to stimulate the formation of new blood vessels.

“ProtheraCytes®” is the trade name for the applicant's isolated CD34+ cells for use in therapeutic applications. ProtheraCytes® are human autologous CD34+ cells expanded according to a GMP automated manufacturing process designed for large clinical scale production. ProtheraCytes® are registered as an ATMP—Advanced Therapy Medicinal Product—within the classification of Tissue Engineered product by the European Medicines Agency. A process for making ProtheraCytes® is set out in Example 1.

“StemXpand®” is the trade name for an incubator system for the automated expansion of cells, such as CD34+ cells. The StemXpand® system is described in U.S. Ser. No. 10/676,705B2.

“StemPack” is the trade name for the single use cell culture kits used for the expansion of ProtheraCytes® in the StemXpand® system.

“StemFeed®” is the trade name for a proprietary medium for expansion of CD34+ cells comprising basal IMDM medium, human plasma and a mix of cytokines.

“microRNAs” (miRNAs) are small non-coding RNAs that influence gene expression.

### Methods of Treating Stroke

The populations of CD34+ cells described herein are provided for use in a method of treating stroke, the method comprising intranasally administering said population of CD34+ cells to a subject.

In an embodiment, the stroke treated by the methods disclosed herein is ischaemic stroke or transient ischaemic attack. In an embodiment, the stroke treated by the methods disclosed herein is ischaemic stroke.

The ischaemic stroke treated by the methods disclosed herein may be thrombotic stroke and/or embolic stroke.

Also provided herein are methods of treating stroke comprising administering intranasally a population of CD34+ cells described herein to a subject.

As discussed elsewhere herein, the CD34+ cells may be obtained from the subject that is to be treated for stroke, i.e. the treatment may involve autologous transplantation of CD34+ cells.

Preferably, the CD34+ cells may be obtained from a source other than the subject to be treated, such as umbilical cord blood or human pluripotent stem cells, i.e. the treatment may involve allogeneic transplantation of CD34+ cells. CD34+ cells obtained from umbilical cord blood (or other allogeneic sources) can be cryopreserved and stored, for example in the clinic, for rapid treatment of future subjects. Therefore, transplantation of allogeneic CD34+ cells is advantageous as it allows for cells to be administered more quickly after the stroke event than use of autologous CD34+ cells.

The population of CD34+ cells may be for use in a method of treating stroke, wherein the CD34+ cells increase the expression of doublecortin (DCX). Doublecortin is required for normal migration of neurons into the cerebral cortex (Gleeson et al). The population of CD34+ cells may be administered intranasally. Expression of doublecortin (DCX) may be increased in the brain. Expression of DXC may be increased in the infarct and/or peri-infarct areas.

Also provided is a population of CD34+ cells for use in a method of treating stroke, wherein the CD34+ cells are administered to a subject intranasally, wherein the CD34+ cells secrete paracrine factors in an area affected by stoke thereby treating stroke. Paracrine factors may include one or more of pro-angiogenic miRNAs (e.g. miRNA 126, 130a, 378, and/or 26a), anti-apoptotic miRNAs (e.g. miRNA 21 and/or 146a), anti-fibrotic miRNAs (133a, 24, 29b, 132), and growth factors such as VEGF. Paracrine factors may be secreted by the population of CD34+ cells in the brain. Paracrine factors may be secreted in exosomes.

Also provided is a population of CD34+ cells for use in a method of treating stroke, wherein the CD34+ cells are administered to a subject intranasally, wherein the CD34+ cells promote angiogenesis in an area affected by stroke, thereby treating stroke. Angiogenesis may be stimulated in the brain. Angiogenesis may be stimulated in the infarct and/or peri-infarct areas.

Also provided is a population of CD34+ cells for use in a method of stimulating angiogenesis in a subject. The method may comprise administering the CD34+ cells to a subject intranasally. The CD34+ cells may be administered to a subject in need of treatment for stroke. Angiogenesis may be stimulated in the brain. Angiogenesis may be stimulated in the infarct and/or peri-infarct areas.

Also provided is a population of CD34+ cells for use in a method of treating stroke, wherein the CD34+ cells are administered to a subject intranasally, and wherein the CD34+ cells promote neurogenesis.

Promotion of neurogenesis may correlate with upregulation of doublecortin (DCX). Neurogenesis may be promoted in the brain. Neurogenesis may be promoted in the infarct and/or peri-infarct areas.

Also provided is a population of CD34+ cells for use in a method of promoting neurogenesis in a subject. The method may comprise administering the CD34+ cells to the subject intranasally. Promotion of neurogenesis may correlate with upregulation of doublecortin (DCX). Neurogenesis may be promoted in the brain. Neurogenesis may be promoted in the infarct and/or peri-infarct areas.

Also provided is a population of CD34+ cells for use in a method of treating stroke, wherein the CD34+ cells are administered to a subject intranasally, and wherein the CD34+ cells promote reduction in infarct size, and/or reduce cell loss in the infarct area and/or peri-infarct area and/or reduce inflammation in the infarct and/or peri-infarct area, thereby treating stroke.

Also provided is a population of CD34+ cells for use in a method of treating stroke, wherein the CD34+ cells are administered to a subject intranasally, and wherein the CD34+ cells promote motor and/or neurological recovery, thereby treating stroke.

Any of the treatments disclosed herein may achieve one or more of the following:
reduce infarct damage
reduce cell loss in infarct and/or peri-infarct areas
reduce the presence of inflammatory cells in an area of the subject affected by ischemia
reduce inflammation in infarct and/or peri-infarct areas
promote neurogenesis in a subject. Neurogenesis may correlate with upregulation of doublecortin (DCX).
promote angiogenesis in a subject. Angiogenesis may be promoted through the secretion of paracrine factors such as VEGF and/or exosomes, and/or any miRNAs disclosed herein and may correlate with upregulation of vascular endothelial growth factor receptor 1 (VEGFR-1).
Reduce impairment or speed up recovery from stroke-induced lesions, such as motor, cognitive, language, visual, olfactory, somatosensory, behavioural, memory and any combination thereof.

CD34+ cells may secrete extracellular vesicles (exosomes), for example, in the brain, which may contain paracrine factors as described herein. Vesicles may be CD63+ extracellular vesicles/exosomes.

Where autologous CD34+ cells are to be used, any of the methods disclosed herein may comprise one or more of the following steps:
administering a hematopoietic growth factor, such as G-CSF, to a subject,
collecting a peripheral blood sample from the subject,
isolating and/or expanding CD34+ cells from the blood sample to obtain a population of CD34+ cells, and/or
administering the CD34+ cells to the subject.

The population of CD34+ cells for use in the invention may be an isolated and/or purified/and/or cultured and/or expanded population of CD34+ cells (eCD34+).

The CD34+ cells may be administered to the subject within 3 hours, 6 hours, 12 hours, 1 day, 2 days, 3 days, 4 days, 5 days, 6 days, 7 days, 8 days, 9 days or 10 days, of the stroke event. Preferably, the CD34+ cells may be administered to the subject within 3 days, or within 4 days, of the stroke event.

### Patient Groups

The population of CD34+ cells for use in the methods disclosed herein are administered to a subject. The subject may be in need of treatment for stroke. The subject may be a mammalian subject, such as human, horse, dog, or cat. The subject may be a human.

### Isolation of CD34+ Cells

The CD34+ cells may be autologous or allogeneic CD34+ cells. The CD34+ cells are preferably allogeneic CD34+ cells obtained from umbilical cord blood.

The umbilical cord blood sample may be obtained fresh or frozen from a cord blood bank after signed informed consent.

The whole blood sample may be obtained following granulocyte-colony stimulating factor (G-CSF) mobilisation. The whole blood sample may be subjected to red blood cell sedimentation.

The blood sample may be subjected to total nuclear cell isolation. Total nuclear cell isolation may follow the gelatin method or alternative method without gelatin via centrifugation. In the gelatin method, the blood sample is mixed with a gelatin solution and hung fora period of time to facilitate red blood cell sedimentation. The red blood cells remaining in the pellet may be mixed with gelatin and hung for a second period of time. Following sedimentation, the supernatant may be centrifuged to pellet the total nuclear cells. Following centrifugation, the CD34+ cells may be purified by immunoselection. Immunoselection may be performed by any known method, for example with the CliniMACS system (Magnetic-Activated Cell Sorting).

Where allogeneic CD34+ cells are used in the invention, the cells may be genetically modified in order to reduce, minimise or avoid host rejection. For example, cells may be modified by techniques and tools known in the art, such as CRISPR-based systems, TALEN, or ZFN, to inhibit the expression of proteins that play a role in transplant rejection, such as HLA/MHC and related proteins. CD34+ cells may be genetically modified to express genes that promote neurogenesis and/or angiogenesis.

Where allogeneic CD34+ cells are used in the invention, further therapeutic agents may be administered in combination with the CD34+ cells. For example, the CD34+ cells may be administered with an immunosuppressive agent, such as cyclosporin A.

### Culture or Expansion of CD34+ Cells

The purified CD34+ cells may be cultured and/or expanded. The CD34+ cells may be cultured and/or expanded for 5 to 12 days. The CD34+ cells may be cultured and//or expanded for 5, 6, 7, 8, 9, 10, 11, or 12 days. The CD34+ cells may be cultured and/or expanded for 8-9 days. The CD34+ cells may be cultured and/or expanded at 37° C. The CD34+ cells may be cultured and/or expanded in a 5% CO2 controlled atmosphere. The CD34+ cells may be cultured and/or expanded in a culture medium comprising cytokines such as interleukin 6 (I1L6), interleukin 3 (IL3), Stem Cell Factor, ThromboPoietin, and Fms-Like Tyrosin kinase 3 Ligand at various concentrations. Cells may be cultured at any suitable concentration, for example 2.5×10⁵ cells/mL.

The invention may be carried out using human autologous or allogeneic CD34+ cells expanded according to a GMP automated manufacturing process designed for large clinical scale production, e.g. ProtheraCytes®.

The CD34+ cells for use in the invention may be a cultured and/or expanded cell population.

The cultured or expanded CD34+ cell population may be produced by the methods disclosed herein.

### Additional Processing Steps

Following the culture or expansion of CD34+ cells as described above, the CD34+ cells may undergo further processing steps, such as, for example, purification and/or immunoselection.

The CD34+ cells may be immunoselected for example using magnetic activated cell sorting. The immunoselected CD34+ cells may then be resuspended in a buffer, for example 15 ml. The buffer may comprise albumin and/or saline and/or PBS. The buffer may comprise or consist of 2%, 3%, 4%, or 5% albumin in saline. In an embodiment, the buffer is 4% albumin in saline. The buffer may be phosphate buffered saline (PBS)/2′)/0 human serum albumin (HSA). The cells may be resuspended in 1-15 ml PBS/2′)/0 HSA. The cell suspension may be conditioned in syringes of 4-5 ml. The CD34+ cell population for use according to the present invention may be provided as a population of cells suspended in buffer. The population of CD34+ cells for use in the invention may be provided as a suspension in, for example, PBS/2′)/0 human serum albumin.

Said processing may be to form a product suitable for use in the invention. For example, ProtheraCytes® are CD34+ cells which have been processed to form a product suitable for use in a method of treatment. ProtheraCytes® are registered as an ATMP (Advanced Therapy Medicinal Product) within the classification of Tissue Engineered product by the European Medicines Agency. A process for making ProtheraCytes® is set out in Example 1.

The CD34+ cells may be cryopreserved via a controlled rate freezing process (1° C./min) with cryoprotectants or solutions such as CryoStor CS10. The cells to be cryopreserved may be centrifuged to obtain a cell pellet. The supernatant may then be removed and cold (2-8° C.) CryoStor medium may be added to the cell pellet to obtain a cell concentration range of 0.5-10×10⁶ cells/ml. The cell suspension may then be incubated at 2-8° C. for 10 minutes. Then, using a controlled rate freezer (−1° C./minute) the temperature of the sample may be lowered to −80° C., before transferring to liquid nitrogen temperatures (below −130° C.).

Alternatively, cells may be frozen using stepwise freezing procedures. One example is a stepwise freezing procedure is as follows: 2 hours at −20° C. followed by 2 hours at −80° C., or 3-4 hours at −80° C. in an isopropanol freezing container. The isopropanol container may be pre-cooled to 2-8° C. Mechanical agitation (flick or tap) of the sample container may be performed after 15-20 minutes at −80° C. Samples should be kept at −80° C. for at least 10 hours. Finally, the samples may be stored at liquid nitrogen temperatures (below −130° C.).

### Populations of CD34+ Cells for Use in the Invention

The population of CD34+ cells for use in the invention may be isolated from a blood sample. The blood sample may be an umbilical cord blood sample or a whole blood sample. Preferably, the blood sample is an umbilical cord blood sample. A population of CD34+ cells that has been isolated from a blood sample may be referred to as an “isolated population of CD34+ cells”.

The population of CD34+ cells for use in the invention may be a cultured and/or expanded and/or purified population of CD34+ cells. Methods for culturing, expanding and purifying CD34+ cells are disclosed herein.

The population of CD34+ cells for use in the invention may have a CD34+ cell viability of at least about 70%, 75%, 80%, 85%, 90%, 95% or 99%. In a preferred embodiment the population of CD34+ cells for use in the methods of the invention have a CD34+ cell viability of at least about 90%.

The population of CD34+ cells for use in the invention may have a CD34+ cell purity of at least about 60%, 65%, 70%, 75%, 80%, 85%, 90%, 95% or 99%. In a preferred embodiment the population of CD34+ cells for use in the methods of the invention have a CD34+ cell purity of at least about 75%.

The population of CD34+ cells for use in the invention may comprise less than or equal to about 40%, 35%, 30%, 25%, 20%, 15%, 10%, 5%, 3%, 2% or 1% monocytes. In a preferred embodiment the population of CD34+ cells for use in the methods of the invention comprise less than or equal to about 15% monocytes.

The population of CD34+ cells for use in the invention may comprise less than or equal to about 40%, 35%, 30%, 25%, 20%, 15%, 10%, 5%, 3%, 2% or 1% granulocytes. In a preferred embodiment the population of CD34+ cells for use in the invention comprises about 5% or less granulocytes.

The population of CD34+ cells for use in the invention may comprise less than or equal to about 40%, 35%, 30%, 25%, 20%, 15%, 10%, 5%, 3%, 2% or 1% lymphocytes. In a preferred embodiment the population of CD34+ cells for use in the invention comprises about 3% or less lymphocytes.

The population of CD34+ cells for use in the invention may comprise at least about 2×10⁶ CD34+ cells, at

least about 3×10⁶ CD34+ cells, at least about 4×10⁶ CD34+ cells, at least about 5×10⁶ CD34+ cells, at
least about 6×10⁶ CD34+ cells, at least about 7×10⁶ CD34+ cells, at least about 8×10⁶ CD34+ cells, at least about 9×10⁶ CD34+ cells, or at least about 10×10⁶ CD34+ cells. Preferably, the population of CD34+ cells for use in the invention comprises at least about 7×10⁶ CD34+ cells.

The population of CD34+ cells for use in the invention may comprise about 2×10⁶ CD34+ cells, about 3×10⁶ CD34+ cells, about 4×10⁶ CD34+ cells, about 5×10⁶ CD34+ cells, about 6×10⁶ CD34+ cells, about 7×10⁶ CD34+ cells, at least about 8×10⁶ CD34+ cells, about 9×10⁶ CD34+ cells, or about 10×10⁶ CD34+ cells. Preferably, the population of CD34+ cells for use in the invention comprises about 7×10⁶ CD34+ cells.

In an embodiment, the CD34+ cell population for use in the invention has one or more or all of the following features:
(i) at least about 75% CD34+ cells, and/or
(ii) about 15% monocytes or lower, and/or
(iii) about 5% granulocytes or lower, and/or
(iv) about 3% lymphocytes or lower.

In an embodiment, the CD34+ cell population for use in the invention has one or more of the following features:
(a) about 7×10⁶ CD34+ cells,
(b) a CD34+ cell viability of at least about 90%,
(c) a CD34+ cell purity of at least about 75%,
(d) about 15% monocytes or lower,
(e) about 5% granulocytes or lower, and
(f) about 3% lymphocytes or lower.

In an embodiment the CD34+ cell population for use in the invention has all of features (a)-(f) listed above.

CD34+ cell viability and purity can be determined by any suitable method known in the art. For example, cell viability and purity can be determined via flow cytometry. Kits enabling reliable enumeration of CD34+ stem cells are commercially available, for example the BD® Stem Cell Enumeration Kit (BD Biosciences). Cell counts may be performed at any stage of the manufacturing process, for example, on WB, before a first round of immunoselection, before expansion, before a second round of immunoselection, at the end of the process as in-process controls or any combination thereof. Cell counts may be performed using the Stem Cell Enumeration kit and the Stem Cell Control kit (both from BD Biosciences, San Jose, CA) and analyzed with a Fluorescence Activated Cell Sorting (FACS) Canto II analyzer (BD Biosciences) and FACS DIVA software following the manufacturer's instructions and ISHAGE guideline (Gratama et al). The proportion of immature CD34+ cells can be determined by analysing CD133+ co-expression as described in Saucourt et al. The percentage of cell impurities may be determined as described in Saucourt et al.

### Expression Characteristics of Populations of CD34+ Cells

The population of CD34+ cells for use in the invention may express VEGF in a culture medium in an amount indicative of biological and/or therapeutic activity or efficacy as described below. The CD34+ cells for use in the methods of the invention may express miRNAs indicative of biological and/or therapeutic activity as described below. The CD34+ cells for use in the methods of the invention may express both VEGF in a culture medium in an amount indicative of biological and/or therapeutic activity or efficacy and miRNAs indicative of biological and/or therapeutic activity as described below.

The population of CD34+ cells for use in the invention may express CD44.

### Determination of VEGF Expression

The population of CD34+ cells of the invention may be assayed to determine VEGF expression levels.

The amount of VEGF expressed by a population of CD34+ cells may be determined by any known means, for example, by western blot, enzyme-linked immunosorbent assay (ELISA), fluorescence-linked immunosorbent assay (FLISA), competition assay, radioimmunoassay, lateral flow immunoassay, flowthrough immunoassay, electrochemiluminescent assay, nephelometric-based assays, turbidometric-based assay, or fluorescence activated cell sorting (FACS)-based assays. Determination of the amount

of VEGF may be by mass spectrometry. Determination of the amount of VEGF may be by radioimmunoassay. Preferably, determination of the amount of VEGF is by ELISA. For example, determining the amount of VEGF expressed by a population of CD34+ cells may involve one or more of the following:
collecting supernatant from CD34+ cell cultures;
storing the supernatant;
measuring VEGF using an ELISA kit e.g. QuantiGlo ELISA Kit (R&D Systems, MN, USA) according to the manufacturer's instructions, for example with the SpectraMax L (Molecular Devices, San Jose, CA USA).

A negative control may be used, for example, a culture medium such as StemFeed® medium. A positive control may be used, for example, Immunoassay Control Set 732 for Human VEGF (R&D Systems). amount of VEGF expressed by a population of CD34+ cells may be determined by measuring the concentration of VEGF released by the cells into a cell culture medium. The amount of VEGF expressed by a population of CD34+ cells may be determined by measuring the concentration of VEGF contained in CD34+ cell derived exosomes. The CD34+ cell culture or a portion thereof may be centrifuged and the concentration of VEGF present in the supernatant determined. For example, approximately 50 mL of supernatant may be obtained and frozen as smaller aliquots. Typically, 50 μL of sample per well in the ELISA assay may be used.

### VEGF Expression of CD34+ Cells

The population of CD34+ cells for use in the methods of the invention may express VEGF in a culture medium in an amount indicative of biological and/or therapeutic activity or efficacy. For example, CD34+ cells for use in the methods of the invention may express VEGF in a culture medium or supernatant of a CD34+ cell culture in an amount of about 1, 5, 10, 20, 25, 50, 75, 100, 150, 155, 160, 165, 170, 175, 180, 185, 190, 195 or 200 pg/ml. The CD34+ cells for use in the methods of the invention may express VEGF in a culture medium or supernatant of a CD34+ cell culture in an amount of at least 25 pg/ml. The CD34+ cells for use in the methods of the invention may express VEGF in a culture medium or supernatant of a CD34+ cell culture in an amount of at least 50 pg/ml. The CD34+ cells for use in the methods of the invention may express VEGF in a culture medium or supernatant of a CD34+ cell culture in an amount of at least 150 pg/ml. If the concentration of VEGF contained in CD34+ cell derived exosomes is measured, the CD34+ cells may express VEGF in the exosomes at any threshold value disclosed herein.

The amount of VEGF expressed by a population of CD34+ cells may be determined by measuring an amount of VEGF expressed as an amount per cell. The CD34+ cell culture or a portion thereof may be centrifuged and the amount of VEGF present in the supernatant determined. The number of CD34+ cells may be counted by any known method, for example flow cytometry, and the amount of VEGF may be expressed as an amount per cell. The CD34+ cells for use in the invention may express VEGF in an amount per cell of at least about 4 fg (fg=femtogram 10⁻¹⁵ g), at least about 10 fg, at least about 15 fg, at least about 20 fg, at least about 25 fg, or at least about 30 fg. The CD34+ cells for use in the invention may express VEGF in an amount per cell of at least about 4 fg.

Detection of miRNA Expression

The population of CD34+ cells of the invention may be assayed to determine miRNA expression. The expression of miRNA in a population of CD34+ cells may be detected by measuring miRNA expression in CD34+ cells and/or in CD34+ cell derived exosomes. miRNA may be isolated from exosomes and/or cells and measured.

Exosomes may be purified by centrifugation of CD34+ cells to remove the cells and cell debris. The resulting supernatant may then be centrifuged again to pellet the exosomes. For example, 50 mL of culture supernatant may be collected, and may be frozen into smaller aliquots. Smaller volumes, such as, 200 μL of sample, may be used for extracting miRNA from the exosomes. The miRNA may then be extracted from the exosomes by any known method, such as the use of a commercially available miRNA extraction kit. miRNA may be extracted from CD34+ cells by any known method, such as the use of a commercially available miRNA extraction kit. One example of a commercially available miRNA extraction kit is the Qiagen® miRNeasy® kit.

The miRNAs may be detected and/or quantified by any known method. For example, the miRNAs may be detected and quantified by Quantitative real-time PCR (RT-qPCR), digital PCR, microarray, and/or high-throughput small RNA-sequencing. One example of a commercially available kit for miRNA detection and quantification is the Qiagen® miRCURY LNA miRNA PCR kit. Suitable primers available from Qiagen® include YP00204230 (miR-21-5p), YP00206023 (miR-26a-5p), YP00204227 (miR-126-3p), YP002046658 (miR-130a-3p), YP00204788 (miR-133a-3p), YP00204688 (miR146a-5p), YP00204536 (miR-199a-3p), YP00205946 (miR-378a-3p), and YP00205448 (miR-590-3p). The qPCR data may be normalized to miR-let7a-5p (YP00205727) values. Relative miRNA expressions may be calculated using the 2-^{′8″8.ct} method.

The miRNAs disclosed herein may be detected by, for example, detecting either a 3p and/or a 5p miRNA strand. For example, the miRNAs disclosed herein may be detected via any of the following strands:

miR126-3p
(SEQ ID NO: 1)
UCGUACCGUGAGUAAUAAUGCG
miR126-5p
(SEQ ID NO: 10)
CAUUAUUACUUUUGGUACGCG
miR130a-3p
(SEQ ID NO: 2)
CAGUGCAAUGUUAAAAGGGCAU
miR130a-5p
(SEQ ID NO: 11)
GCUCUUUUCACAUUGUGCUACU
miR21-3p
(SEQ ID NO: 16)
CAACACCAGUCGAUGGGCUGU
miR21-5p
(SEQ ID NO: 3)
UAGCUUAUCAGACUGAUGUUGA
miR26a-3p
(SEQ ID NO: 17)
CCUAUUCUUGGUUACUUGCACG
miR26a-5p
(SEQ ID NO: 4)
UUCAAGUAAUCCAGGAUAGGCU
miR378a-3p
(SEQ ID NO: 5)
ACUGGACUUGGAGUCAGAAGGC
miR378a-5p
(SEQ ID NO: 12)
CCUCCUGACUCCAGGUCCUGUGU
miR146a-3p
(SEQ ID NO: 18)
CCUCUGAAAUUCAGUUCUUCAG
miR146a-5p
(SEQ ID NO: 6)
UGAGAACUGAAUUCCAUGGGUU
miR199a-3p
(SEQ ID NO: 7)
ACAGUAGUCUGCACAUUGGUUA
miR199a-5p
(SEQ ID NO: 13)
CCCAGUGUUCAGACUACCUGUUC
miR590-3p
(SEQ ID NO: 8)
UAAUUUUAUGUAUAAGCUAGU
miR590-5p
(SEQ ID NO: 14)
GAGCUUAUUCAUAAAAGUGCAG
miR133a-3p
(SEQ ID NO: 9)
UUUGGUCCCCUUCAACCAGCUG
miR133a-5p
(SEQ ID NO: 15)
AGCUGGUAAAAUGGAACCAAAU
miR24-1-5p
(SEQ ID NO: 19)
UGCCUACUGAGCUGAUAUCAGU
miR24-2-5p
(SEQ ID NO: 20)
UGCCUACUGAGCUGAAACACAG
miR24-3p
(SEQ ID NO: 21)
UGGCUCAGUUCAGCAGGAACAG
miR132-5p
(SEQ ID NO: 22)
ACCGUGGCUUUCGAUUGUUACU
miR132-3p
(SEQ ID NO: 23)
UAACAGUCUACAGCCAUGGUCG
miR29b-1-5p
(SEQ ID NO: 24)
GCUGGUUUCAUAUGGUGGUUUAGA
miR29b-2-5p
(SEQ ID NO: 25)
CUGGUUUCACAUGGUGGCUUAG
miR29b-3p
(SEQ ID NO: 26)
UAGCACCAUUUGAAAUCAGUGUU

miRNA Expression of CD34+ Cells for Use in the Methods of the Invention

The population of CD34+ cells for use in the method of the invention may express miRNA indicative of biological and/or therapeutic activity or efficacy. For example, the population of CD34+ cells for use in the method of the invention may express one or more or each of miR126, miR130a, miR21, miR26a, miR378a, miR146a, miR199a, miR590, miR133a, miR-24, miR29b, and miR132.

The population of CD34+ cells for use in the invention may express one or more or each of miR126, miR130a, miR21, miR26a, and miR378a.

The population of CD34+ cells for use in the invention may express one or more or each of miR21, miR26a, and miR378a. The population of CD34+ cells for use in invention may express miR146a and/or miR21. The population of CD34+ cells for use in the invention may express miR199a and/or miR590. The population of CD34+ cells for use in the invention may express miR133a. The miRNA may be expressed in the cell and/or in exosomes. CD34+ cells for use in the invention may secrete exosomes containing one or more (for example, all) of the miRNAs disclosed herein.

### Delivery, Doses and Dosage Regimes

The population of CD34+ cells for use in the invention may be provided as a sterile suspension of cells. The sterile suspension is preferably aqueous. The sterile suspension is preferably isotonic. The sterile suspension may further comprise pharmaceutically acceptable carriers, diluents or wetting agents. The population of CD34+ cells may be provided as a pharmaceutical composition comprising one or more additional agents such as pharmaceutically acceptable carriers, buffers, diluents or wetting agents. Pharmaceutical compositions disclosed herein may include a population of CD34+ cells and a buffer. The buffer may comprise albumin and/or saline and/or PBS. The buffer may comprise or consist of 2%, 3%, 4%, or 5% albumin in saline. In an embodiment, the buffer is 4% albumin in saline. The buffer may be phosphate buffered saline (PBS)/2′)/0 human serum albumin (HSA). The CD34+ cell population for use according to the present invention may be provided as a population of cells suspended in buffer. The population of CD34+ cells for use in the invention may be provided as a suspension in, for example, PBS/2′)/0 human serum albumin.

The population of CD34+ cells for use in the invention is administered intranasally to a subject.

For example, an administration protocol may comprise one or more, for example all, of the following steps:
1. A vial containing CD34+ cells is taken from the liquid nitrogen storage and placed on ice.
2. The vial is then thawed in a hot water bath at 37° C.
3. When the tube is nearly thawed (small ice bloc visible), the tube is disinfected and transferred to a biosafety cabinet (BSC).
4. The cell suspension is transferred to a 50 mL tube
5. 20 mL of RPMI is added to the tube and centrifuged at 400 g for 5 min at room temperature.
6. After centrifugation, the supernatant is carefully discarded under the BSC.
7. The cell pellet is then resuspended in the buffer for injection (2% HSA in PBS or alternative).
8. The cell suspension is drawn up a syringe and the syringe is connected to a nozzle or a catheter for intranasal delivery.
9. Optionally, a solution of hyaluronidase is sprayed inside the nostrils, for example about 30 min prior to the cell administration.
10. 1-2 mL of cell suspension is sprayed (nozzle) or injected (catheter) per nostril of the patient, who is resting in a supine position.
11. After cell administration, the patient remains in the supine position for 10 min.

The number of CD34+ cells administered per dose may be about 1×10⁶, about 2×10⁶, about 3×10⁶, about 4×10⁶, about 5×10⁶, about 6×10⁶, about 7×10⁶, about 8×10⁶, about 9×10⁶, or about 10×10⁶. Preferably, the number of CD34+ cells administered per dose is about 7×10⁶. Cells may be counted by any suitable method, such as flow cytometry. Kits enabling reliable enumeration of CD34+ stem cells are commercially available, for example the BD® Stem Cell Enumeration Kit (BD Biosciences).

The number of CD34+ cells administered per dose may be at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶. Preferably, the number of CD34+ cells administered per dose is at least about 7×10⁶.

The CD34+ cells are administered intranasally. The administration of the CD34+ cells may comprise administering CD34+ cells to one or both nostrils. The volume administered per nostril may be about 0.1 ml, about 0.2 ml, about 0.4 ml, about 0.5 ml, about 0.75 ml, about 1 ml, about 2 ml, about 3 ml, about 4 ml, about 5 ml, about 10 ml, or about 15 ml. In a preferred embodiment, the volume administered per nostril is about 1 ml.

The total volume of CD34+ cells administered per dose may therefore be 0.2 ml, about 0.4 ml, about 0.8 ml, about 1 ml, about 1.5 ml, about 2 ml, about 4 ml, about 6 ml, about 8 ml, about 10 ml, about 20 ml, or about 30 ml.

One or more doses of isolated CD34+ cells may be given to a subject. For example, 1 dose, 2 doses, 3 doses, 4 doses, or 5 doses of isolated CD34+ cells may be given to a subject. In a preferred embodiment, 1 dose of isolated CD34+ cells is given to a subject. Successive doses may be separated by at least one month, for example 1 month, 2 months, 3 months, 4 months, 5 months, 6 months, or longer.

### INCORPORATION BY REFERENCE

All documents cited herein are incorporated by reference to the fullest extent permitted by law.

### Examples

### Example 1: Obtaining CD34+ Cells

### Patient Samples, Healthy Donors and Cell-Production Centers

AMI patients and healthy male volunteers were enrolled in this study after approval by the French regulatory agency Agence Nationale de Sécurité du Médicament et des produits de sante and the regional ethics committee. All participants provided signed informed consent. Each participant first underwent daily subcutaneous (s.c.) administration of 10 μg/kg per day G-CSF (Lenograstim) for 4 days. A whole blood (WB) sample of 250-440 mL±10 mL was withdrawn in the morning of the fifth day by simple venous puncture and collected in a blood bag and immediately shipped at ambient temperature to the Cell Production Centre. The manufacturing process was started on the sixth day, after overnight storage of the WB sample at 4° C.-8° C. C, using the StemXpand® automated integrated system and Stem Pack® disposable kits developed by CellProthera®.

CD34+ ces from Umbilical Cord Blood

Fresh or frozen umbilical cord blood was received from cord blood banks after the participants provided signed informed consent.

### ProtheraCytes® Preparation

Starting from the initial WB sample, red blood cell (RBC) sedimentation was performed for total nuclear cell (TNC) isolation using the gelatin method or alternative method without gelatin using centrifugation. Briefly for the gelatin method, 250-440 mL of WB/phosphate-buffered saline 1:1 solution (PBS; Macopharma, Mouvaux, France) was mixed with the same volume of (250-440 mL) of 4% gelatin (Gelofusine, BBraun, Melsungen, Germany) in two 600-ml transfer bags, which were hung for 20 minutes to facilitate RBC sedimentation. RBCs remaining in the pellet were again mixed with 4% gelatin for a second 20-minute sedimentation period. The two supernatants were pooled and centrifuged at 400 g for 10 minutes at room temperature to pellet the TNC, from which basal (b)-CD34+ stem cells (SCs) were purified using the CliniMACS system (Magnetic-Activated Cell Sorting, Miltenyi Biotec, Bergisch Gladbach, Germany). The bag containing purified b-CD34+SC suspension or thawed frozen healthy donor (FHD) CD34+ cells (Lonza), was immediately connected to the machine kit to undergo a 9-day culture period in our proprietary StemFeed® medium into the StemXpand® incubator, in which the expansion steps are automatically programmed and controlled: first, predetermined volumes of StemFeed® culture medium, cytokine mix (composed of interleukin IL6, IL3, Stem Cell Factor,

ThromboPoietin, and Fms-Like Tyrosin kinase 3 Ligand at various concentrations), and the CD34+ SCs were successively distributed into the dedicated culture bag placed on the agitator contained in the device incubator. The bag was then gently agitated for 30 seconds to disperse the cell mixture, which was then incubated at 37° C. in a 5% CO2-controlled atmosphere for a 8-9-day cell expansion period, without any further intervention. At the end of incubation, the cell suspension was dispersed by gentle agitation, followed by adjustment of the agitator tray to an 80° inclination to facilitate distribution of the cell suspension into two collection bags in equal volumes. Samples were collected at day 0 and day 7 to analyze sterility alter dispersing the cell suspension and 50° inclination of the agitator tray.

At the end of the 8 to 9-day period, the culture product was collected, centrifuged, and immunoselected using the CliniMACS system for the purification of expanded (e)CD34+SC, which constituted the final product (ProtheraCytes®) once resuspended in 15 ml PBS/2′)/0 human serum albumin (HSA) and conditioned in three syringes of 5 ml each.

### Example 2

This example describes in vivo assessments of efficacy of treatment of ischaemic stroke by administering CD34+ cells by various routes.

Human CD34+ cells were expanded and purified as previously described (Saucourt et al., 2019). The source of the CD34+ cells can be autologous (from the same patient) or allogeneic (from an adult donor or cord blood, preferably cord blood).

The rat model investigated was the one-hour middle cerebral artery occlusion (MCAO) ischaemia with reperfusion model, which is an established ischaemic stroke preclinical model.

The treatment and control groups were allocated as follows:

| | |
|---|---|
| Objectives | Evaluate the behavioural efficacy of ProtheraCytes ® |
| | cells in an experimental stroke model |
| Model | Rat MCAO ischemia reperfusion model |
| Treatment/ | 1. ProtheraCytes ® (intra-arterial treatment group) |
| control | 2. ProtheraCytes ® (intracerebral treatment group) |
| Groups | 3. ProtheraCytes ® (intranasal treatment group) |
| | 4. Saline control (intra-arterial vehicle treatment group) |
| | 5. Saline control (intracerebral vehicle treatment group) |
| | 6. Saline control (intranasal vehicle treatment group) |
| Study size | n = 10/group, total n = 60 rats |

### General Procedures

1. All animals (250 g male, about 8 weeks old, Sprague-Dawley rats) were initially tested behaviourally in elevated body swing test (EBST) and neurological test to obtain baseline behaviours.
2. Next, ail animals underwent the one-hour middle cerebral artery occlusion (MCAO) and reperfusion model to induce stroke.
3. Animals were then tested again in EBST and neurological tests to assess successful stroke induction. Only animals that exhibited significant behavioural deficits in both EBST (75% bias) and neurological tests (2.5 neuroscore) were subsequently enrolled in the next set of procedures.
4. Animals were next randomly assigned to transplantation of ProtheraCytes® at 3 days post-stroke either intra-arterially (3 million cells in 1 ml vehicle; n=10), intracerebrally (300 k cells in 10 μI vehicle; n=10), or intranasally (1 million cells in 36 μI vehicle; n=10) or vehicle alone (saline intra-arterially, intracerebrally, or intranasally; n=30).
5. At specific time points post-stroke (days 7, 14, and 28), the animals were again tested in EBST and neurological tests.
6. Following behavioural testing at day 28, behavioural data were analysed, and animals were sacrificed for histological analyses of the infarct area, peri-infarct cell loss, inflammation, neurogenesis, and angiogenesis.
7. Detailed behavioural tests, stroke protocol, and transplant protocol are provided below.

### Stroke Surgery

Stroke surgery was performed using the middle cerebral artery (MCAO) technique. Animals were anesthetized with a mixture of 1% to 2% isoflurane in nitric oxide/oxygen (69%/30%) via a face mask, and body temperature was maintained at 37±0.3° C. during the surgical procedures. A midline skin incision was made in the neck with subsequent isolation of the left common carotid artery, the externat carotid artery (ECA), and internai carotid artery. Thereafter, a 4-0 monofilament nylon suture (15.0-17.0 mm) was advanced from the common carotid artery bifurcation until it blocked the origin of the MCA. The skin

incision was closed with surgical clips. Animals were allowed to recover from anaesthesia during the 1-hour MCAO. At 1 hour after MCAO, animals were re-anesthetized, and reperfusion commenced with the withdrawal of the nylon suture. We have standardized the MCAO model, with stroke animals showing
≥80% reduction in regional cerebral blood flow (CBF) during the occlusion period as determined by laser Doppler (Perimed, Periflux System 5000). For baseline regional CBF measurement, the laser Doppler
probe was placed over the right frontoparietal cortical area supplied by the MCA. We also have found no significant differences in physiological parameters, including PaO2, PaCO2, and plasma pH measurements. Rats that reached >75% biased swing activity during occlusion were enrolled in the study. Thereafter, surgical incisions were closed, and animals were allowed to recover from anaesthesia.

### Transplantation Protocol

At 3 days after MCAO, animals were randomly assigned to receive transplantation of ProtheraCytes® at either intravascularly (IV) (3 million cells in 1 ml vehicle; n=10), intracerebrally (IC) (300 k cells in 10 μI vehicle; n=10), or intranasally (IN) (1 million cells in 36 μI vehicle; n=10) or vehicle alone (saline intra-arterially, intracerebrally, or intranasally; n=30). was via the carotid artery. Since the animals receive right MCAO, animals also received infusion of cells/saline into the carotid artery. Animals were anesthetized with a mixture of 1% to 2% isoflurane in nitric oxide/oxygen (69%/30%) via a face mask, and body temperature was maintained at 37±0.3° C. during the surgical procedures. Infusion of cells/saline was performed via a bolus injection delivered from a 25 G needle of a 1-mL syringe inserted into the carotid artery. The syringe was filled with cells/saline. After dosing, the needle was removed and the carotid artery pressed for 30 seconds to prevent any bleeding, and the skin wound reclosed with surgical clips.

For IC transplantation, stereotaxic surgery targeted the right striatum. All surgical procedures were conducted under aseptic conditions. Animals were anesthetized with a mixture of 1% to 2% isoflurane in nitric oxide/oxygen (69%/30%) via a face mask, and body temperature was maintained at 37±0.3° C. during the surgical procedures. Once deep anaesthesia was achieved (by checking for pain reflexes), hair was shaved around the area of surgical incision (skull area) with enough border to prevent contaminating the operative site, followed by two surgical germicidal scrubs of site, and draping with sterile drapes. The animal was then fixed to a stereotaxic apparatus (Kopf Instruments). A 26-gauge Hamilton syringe was then lowered into the small burred skull opening (transplant coordinates were adjusted to correspond with the striatal area adjacent to the infarcted site: 0.5 mm anterior and 1.0 mm lateral to bregma and 4.0 mm, 3.5 mm, and 3.0 mm below the durai surface). Within this single needle pass, 3 deposits of the test article (100,000 cells in 3 μI per deposit or a total of 300,000 cells in 9 μI of saline for 3 deposits) were made.

The target area is the media) striatum which corresponds to the peri-infarcted striatal area, based on previously established target sites for similar stereotaxic implants. Each deposit consists of 100,000 viable cells in 3 μI volume infused over a period of 3 minutes. Following an additional 2-minute absorption time, the needle was retracted and the wound closed with stainless steel wound clips. A heating pad and a rectal thermometer allow maintenance of body temperature at about 37° C. throughout surgery and following recovery from anaesthesia.

For IN transplantation, animals were held with a hand grip that allowed the animals to recline on their backs white immobilizing the skull, and the nose drop containing the substance/cell suspension was carefully placed on one nostril allowing it to be snorted naturally, and then the other nostril. One hundredunits of hyaluronidase (Sigma-Aldrich Chemie GmbH, H3506) dissolved in 24 μL sterile PBS was administered to the rat nostrils (6 μL/nostril, repeat once after 2 min) 30 min prior to the administration of ProtheraCytes® or vehicle. 1 million cells resuspended in 36 μL were applied to each rat using the same method as described for hyaluronidase, white control group received the same amount of vehicle only.

### Behavioural Tests

The behavioural tests include the Elevated Body Swing Test (EBST) and the Bederson neurological test. EBST is a measure of asymmetrical motor behaviour that does not require animal training or drug injection. The rat was held in the vertical axis approximately 1 inch from the base of its tail and then elevated to an inch above the surface on which it had been resting. The frequency and direction of the swing behaviour was recorded over 20 tait elevations. A swing was counted when the head of the rat moved more than 100 from the vertical axis to either side. Normally, intact rats display a 50% swing bias, that is, the same number of swings to the left and to the right. A 75% swing bias toward one direction was used as criterion of motor deficit. The total number of swings made to the biased side was added per animal and divided by 20, providing the average number of biased swings per individual animal.

The Bederson neurological test is an evaluation of the animal's sensorimotor function, which consists of three distinctive tasks, performed over approximately 10 minutes per rat. Each task was conducted sequentially and was each scored 0 to 3. These three evaluations included: 1. contralateral hind limb retraction; 2. bilateral forepaw grasp; and 3. beam walking ability.

Contralateral hind limb retraction measures the ability of the animal to replace the hind limb after it is displaced laterally by 2 to 3 cm. Grades are as follows: 0 for immediate smooth replacement, 1 for slow replacement, 2 for partial rigid replacement, and 3 for no replacement.

Bilateral forepaw grasp measures the ability of a rat to hold onto a 2-mm diameter steel rod. Grade 0 is used for rats with normal forepaw grasping behaviour, 1 for rapid grasping but with rigidity, 2 for slow grasping with rigidity, and 3 for a rat unable to grasp with the forepaw.

Beam walking ability uses a beam apparatus that is 80 cm long with a flat surface of 2-4 cm width resting at least 40 cm above the table/surface top on two poles. The animal is placed at one end of the beam then the ability to traverse the beam and reach the other end is assessed. The grades are as follows: 0 for a rat that easily traverses the beam, 1 if the rat slowly traverses the beam, 2 for partially traversing the beam but falls off, and 3 fora rat unable to stay on the beam for 10 seconds.

The scores from all three tests were added to give a total neurologic deficit score (maximum possible score is 9 with mean composite neurologic score of 3). A score of 2.5 was set as a criterion to be considered a “stroke” animal.

### Results

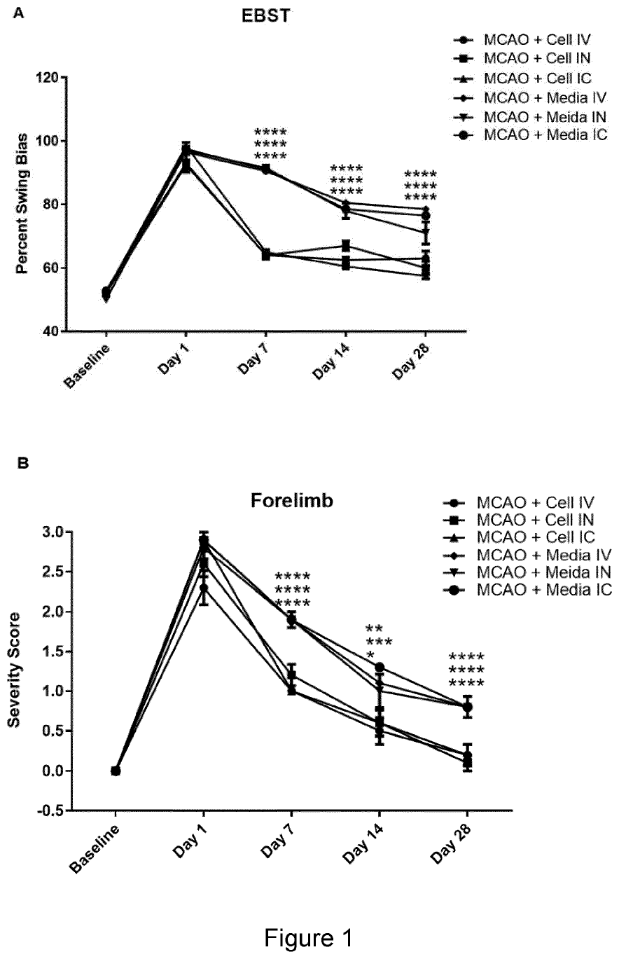

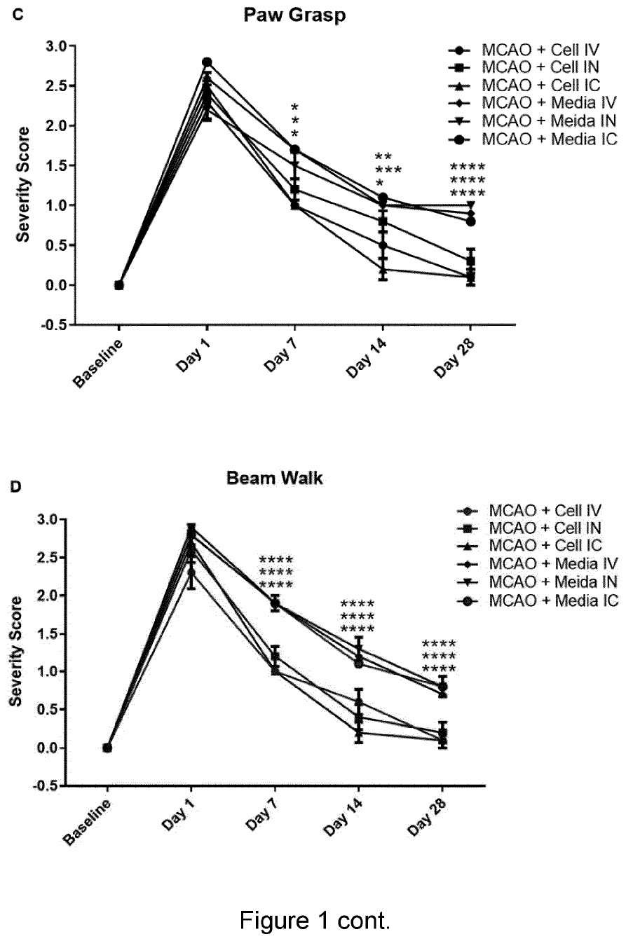

The results of the behavioural tests showed that MCAO rats transplanted with ProtheraCytes® via ail three routes of administration (IV, IC, and IN) had significant improvement for ail the behavioural tests conducted compared to the vehicle control MCAO rats (FIG. 1A-D). Surprisingly, the intranasal administration of ProtheraCytes® had the same efficacy as intracerebral administration. The intracerebral route delivers ProtheraCytes® directly to the peri-infarct area through stereotactic surgery maximizing the engraftment of the cells around the lesion site, whereas the intranasal route delivers cells along the olfactory and trigeminal pathways leading to a disperse biodistribution of cells throughout the central nervous system. So, it was surprising to see that ProtheraCytes® delivered through the intranasal route also engrafted in the peri-infarct area and had similar efficacy as the intracerebral route. In most cases, the treated groups improved to the point of returning to baseline values before the ischaemic lesion (FIG. 1A-D). There were no observable adverse behavioural effects in any of the cell-transplanted stroke animals.

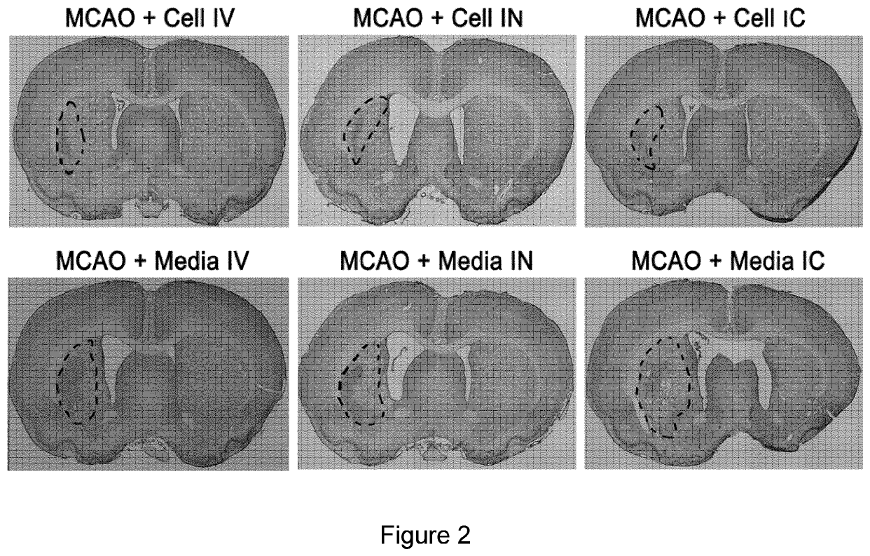

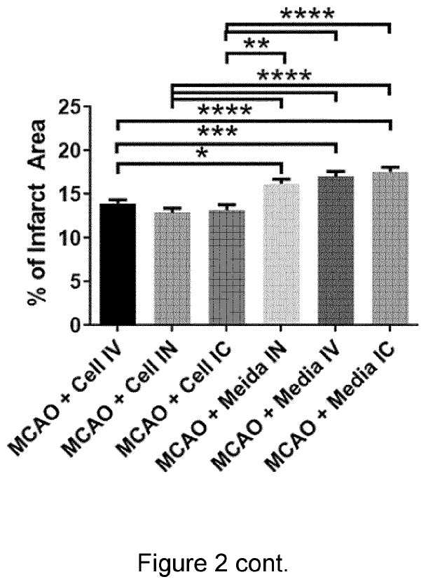

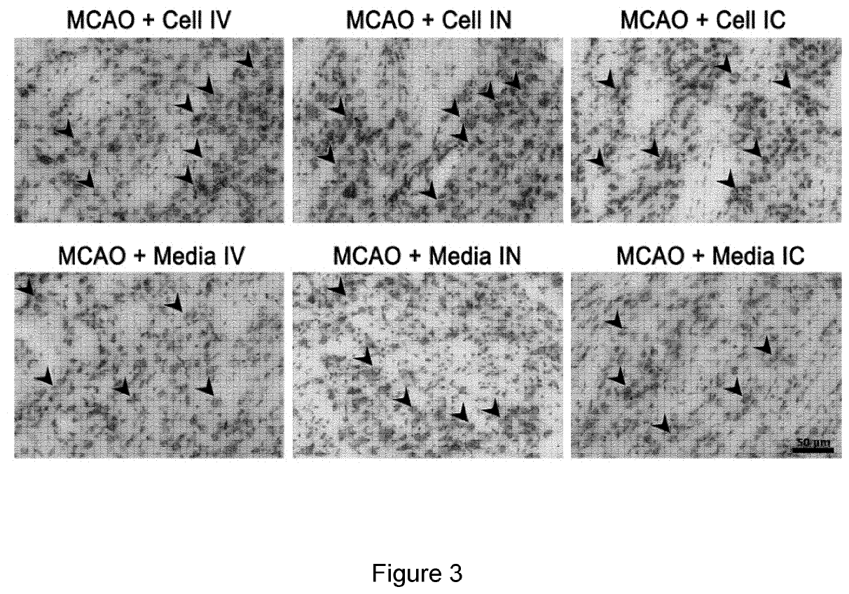

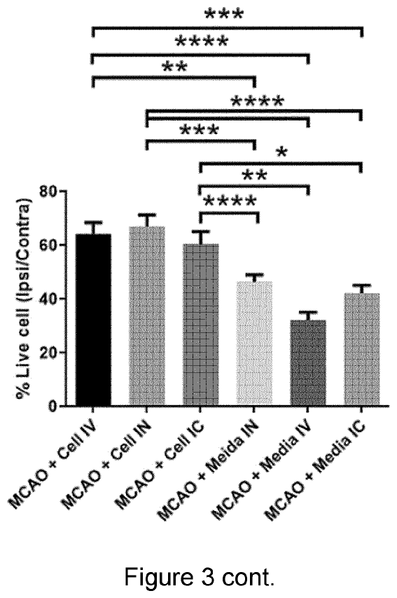

MCAO rats transplanted with ProtheraCytes® via ail three routes of administration had a significant reduction in the infarct area compared to vehicle control MCAO animals (FIG. 2). MCAO rats transplanted with ProtheraCytes® via ail three routes of administration also had a significant reduction of cell loss in the peri-infarct area compared to vehicle control animals (FIG. 3).

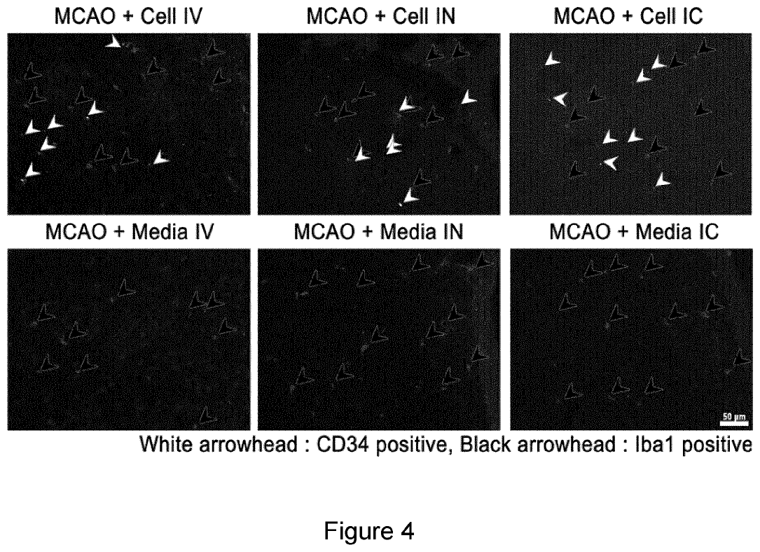

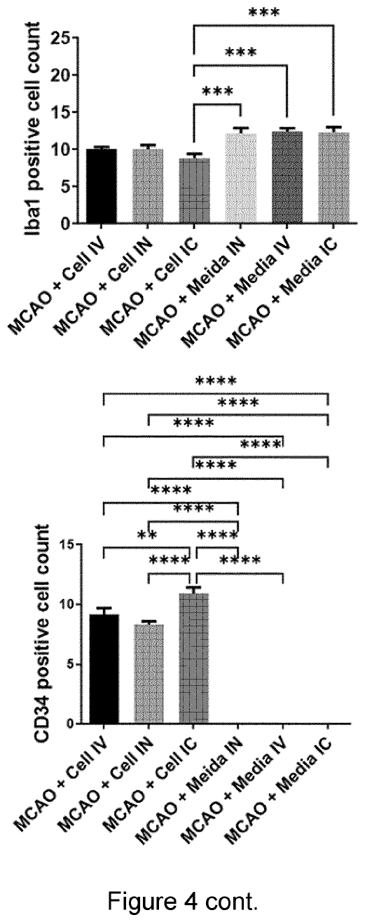

MCAO rats transplanted with ProtheraCytes® via ail three routes of administration also showed a reduction of inflammation (lba-1 labeled inflammatory cells) compared to vehicle control animals (FIG. 4). Survival and engraftment of implanted ProtheraCytes® was detected with ail three routes of administration (FIG. 4).

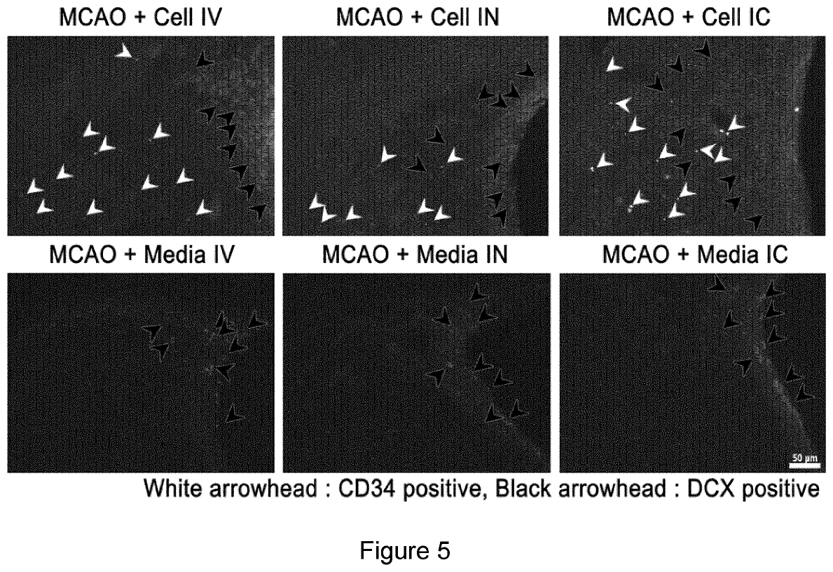

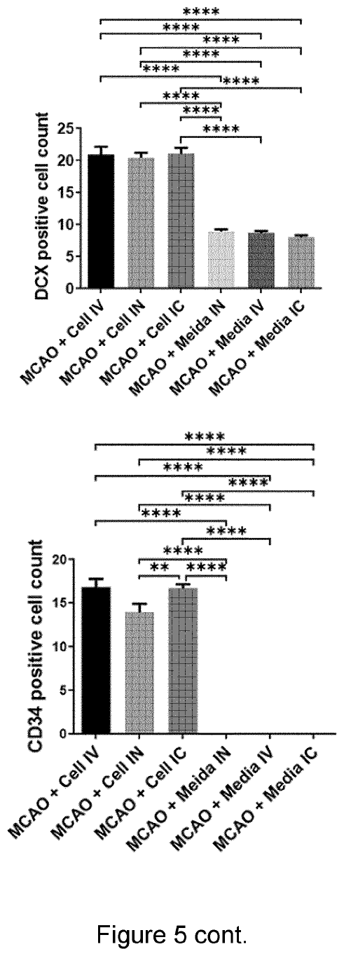

MCAO rats transplanted with ProtheraCytes® via ail three routes of administration showed significant upregulation of neurogenesis (doublecortin DCX) compared to vehicle control animals (FIG. 5).

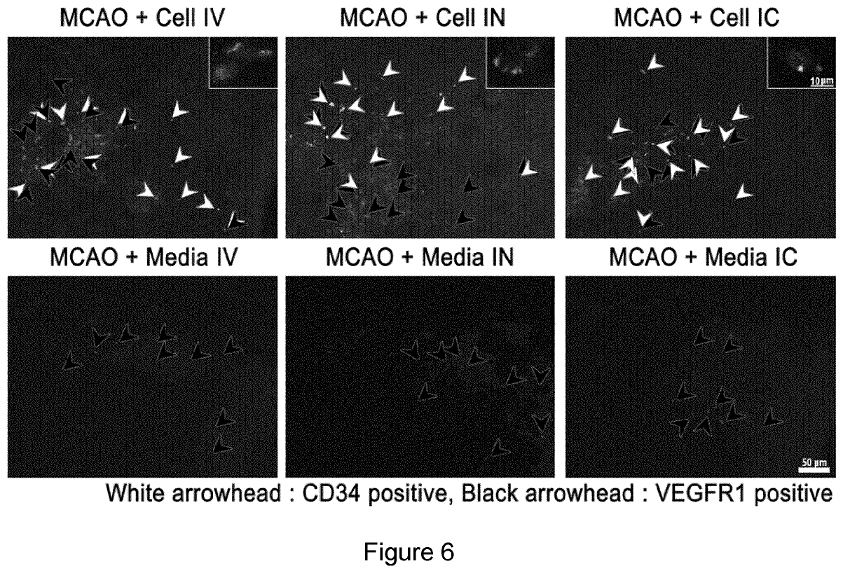

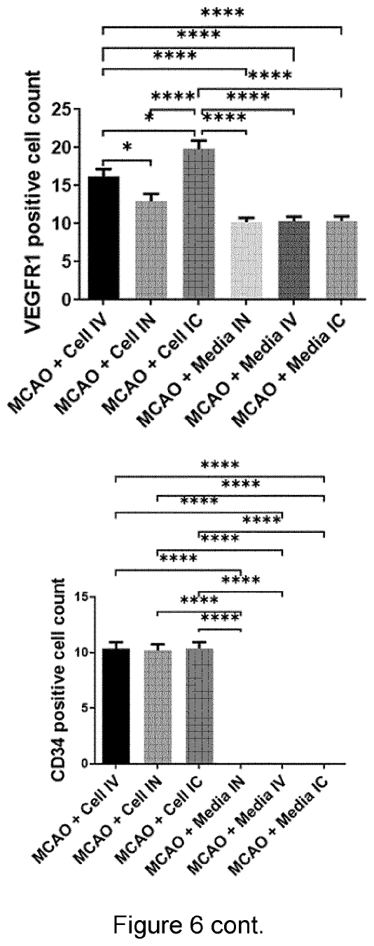

MCAO rats transplanted with ProtheraCytes® via ail three routes of administration had increased angiogenesis (VEGFR1 positive cells) compared to vehicle controls MCAO rats (FIG. 6). Increased angiogenesis was observed close to the implanted ProtheraCytes®, supporting the mechanism of action of CD34+ cells, which promote angiogenesis through the secretion of paracrine factors such as VEGF and exosomes containing miRNAs (FIG. 6).

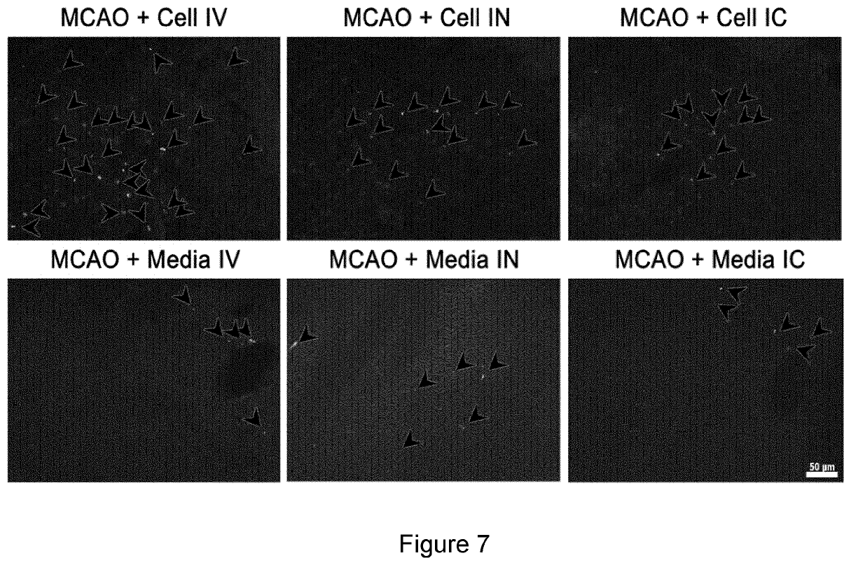

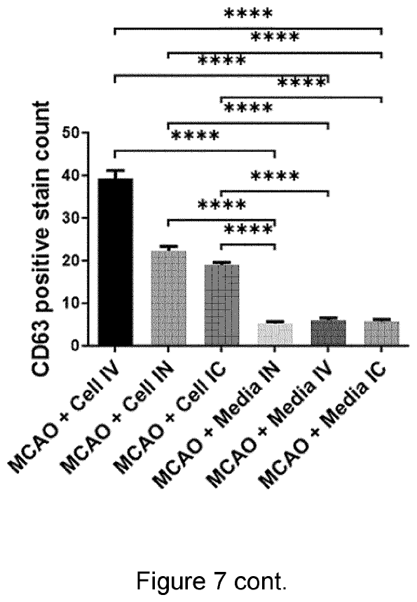

MCAO rats transplanted with ProtheraCytes® via ail three routes of administration, had significantly higher number of extracellular vesicles (CD63+ exosomes) than vehicle control animals (FIG. 7). This important finding shows that ProtheraCytes® secrete extracellular vesicles in vivo and supports results observed in vitro where CD34+ cells are shown to secrete exosomes with pro-angiogenic and anti-apoptotic properties (Example 3). This finding provides further evidence of the mechanism of action of

ProtheraCytes®, showing that cells promote neurogenesis and angiogenesis in the stroke brain through the secretion of these CD63+ extracellular vesicles/exosomes.

This is the first time that intranasal administration of CD34+ cells for the treatment of ischaemic stroke model has been reported. Intranasal administration of CD34+ cells significantly reduced stroke-induced behavioural impairments with the corresponding histological improvements.

### Example 3: Measurement of VEGF Expression and Exosomal miRNA Expression by ProtheraCytes®

### Methods

### Cell Culture

ProtheraCytes® were obtained after expansion of mobilized CD34+ cells from AMI patients (EXCELLENT Phase I/IIb clinicat trial NCT02669810) and from Frozen Healthy Donors (FHD) CD34⁺ purified cells (Lonza, NC, USA) as previously described (Saucourt et al., 2019).

### VEGF Quantification

Supernatants from AMI patients and FHD ProtheraCytes® were collected after 9 days of expansion in StemFeed® cell culture medium (Eurobio, France) and stored at −80° C. until analysis. Supernatants were thawed and VEGF levels were measured using the Human VEGF QuantiGlo ELISA Kit (R&D Systems, MN, USA) according to the manufacturer's instructions with the SpectraMax L (Molecular Devices, San Jose, CA USA). The Immunoassay Control Set 732 for Human VEGF (R&D Systems) was used as a positive control and StemFeed® medium as a negative control.

### In Vitro Tube Formation Assay

2.5×10⁴ serum-starved overnight Human umbilical Vein Endothelial Cells (HUVECs) were seeded with ProtheraCytes® supernatant or StemFeed® medium as negative control into 48-well plates that had been coated with 150 μL of growth-factor—reduced Matrigel™ (Corning, Arizona, US). Tube formation was examined by phase-contrast microscopy 6 hours later and the number of tubes was quantified in each condition in triplicate.

### Exosome Isolation

ProtheraCytes® were seeded in a serum-free medium (DMEM medium) in a bioreactor for extracellular vesicle release by exerting a controlled mechanical stimulation on cells for 30 minutes to 2 hours according to Everzom's proprietary method. The size distribution and concentration measurements of ProtheraCytes®-derived exosomes were performed using the Nanosight (NS300, Malvern, UK). The exosome membrane markers were quantified by flow cytometry using the MACSPlex Exosome kit (Miltenyi).

For miRNA analysis, ProtheraCytes® were cultured at the concentration of 2.5×10⁵ cells/mL in StemSpan-AOF (StemCell Technologies, BC Canada) supplemented with cytokines for 40 hours. Then, cells were collected by centrifugation at 400 g for 10 minutes; and exosomes were purified from the supernatant by precipitation using the ExoQuick-TC™ kit (System Biosciences, CA, USA) according to the manufacturer's instructions. After isolation, exosomes were characterized by flow cytometry using the ExoStep™ kit with a bead-bound anti-CD63 capture, anti-CD81 and anti-CD34 antibodies (ImmunoStep, Spain) confirming the identity of the exosomes secreted by ProtheraCytes®.

### MicroRNA Quantification

Total RNA from ProtheraCytes® and their secreted exosomes collected from 7 AMI patients were isolated respectively using a miRNeasy Tissue/cells Advanced MiniKit and a miRNeasy Serum/Plasma Advanced Kit (QiAGEN, France) according to the manufacturer's protocols. RNA isolated from exosomes and cells was reverse transcribed to cDNA using the miCURY LNA RT Kit (QIAGEN, France). UniSp6 RNA spike-in controls were added during cDNA synthesis to ensure the quality of the experiment. Real-time qPCR amplifications were performed for each RT reaction. Reactions were performed according to the manufacturers' instructions using a miRCURY LNA miRNA SYBR Green PCR Kit (QIAGEN, France) with the Bio-Rad CFX96™ Real time PCR Detection System (BioRad Laboratories, France). All primer sets were custom designed by the supplier. Primers used were miR-21-5p (YP00204230), miR-26a-5p (YP00206023), miR-126-3p (YP00204227), miR-130a-3p (YP002046658), miR-133a-3p (YP00204788), miR146a-5p (YP00204688), miR-199a-3p (YP00204536), miR-378a-3p (YP00205946), and miR-590-3p (YP00205448). The qPCR data were normalized to miR-let7a-5p (YP00205727) values. Relative miRNA expressions were calculated using the 2-^{′8″8.ct} method.

### Results

### Secretion of VEGF as a Potency Test

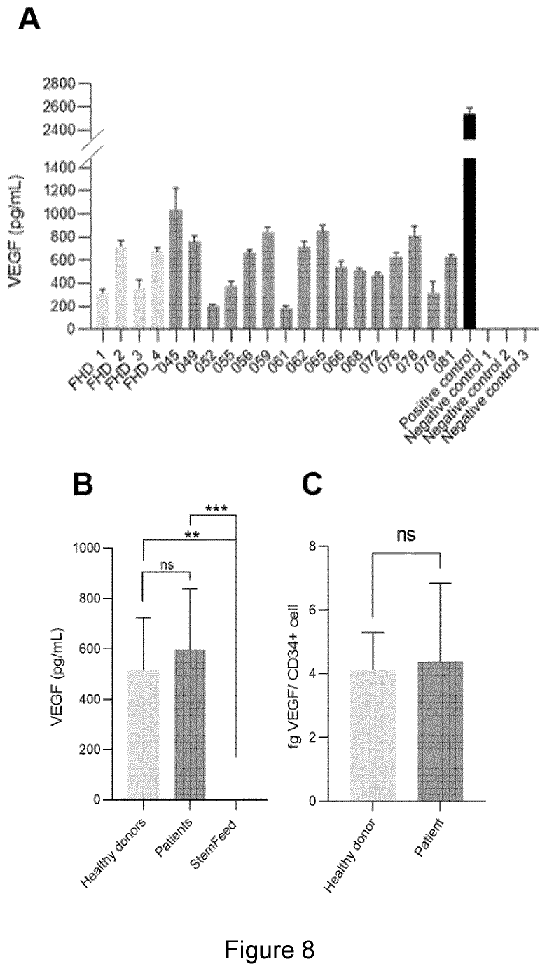

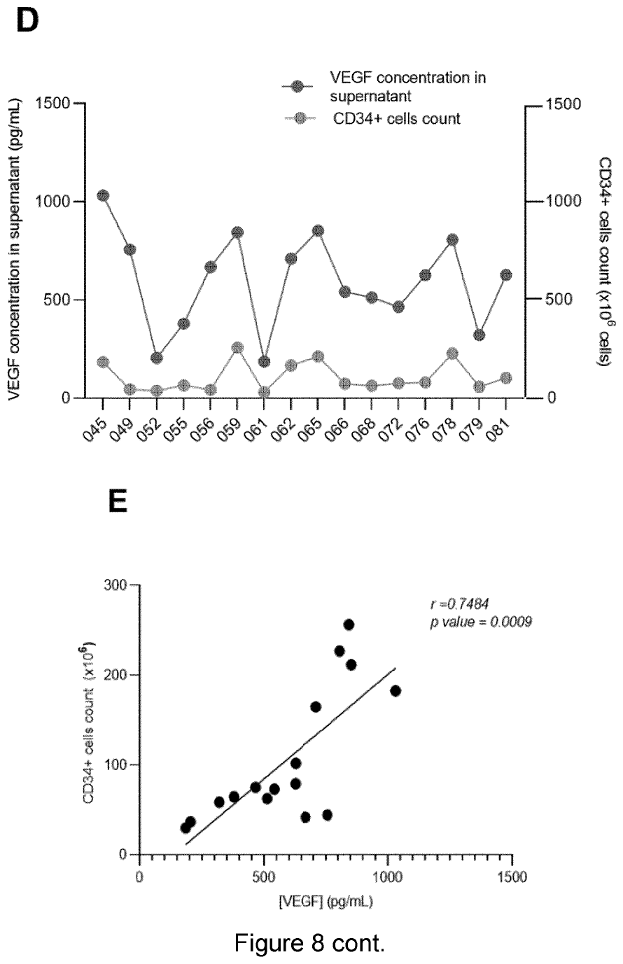

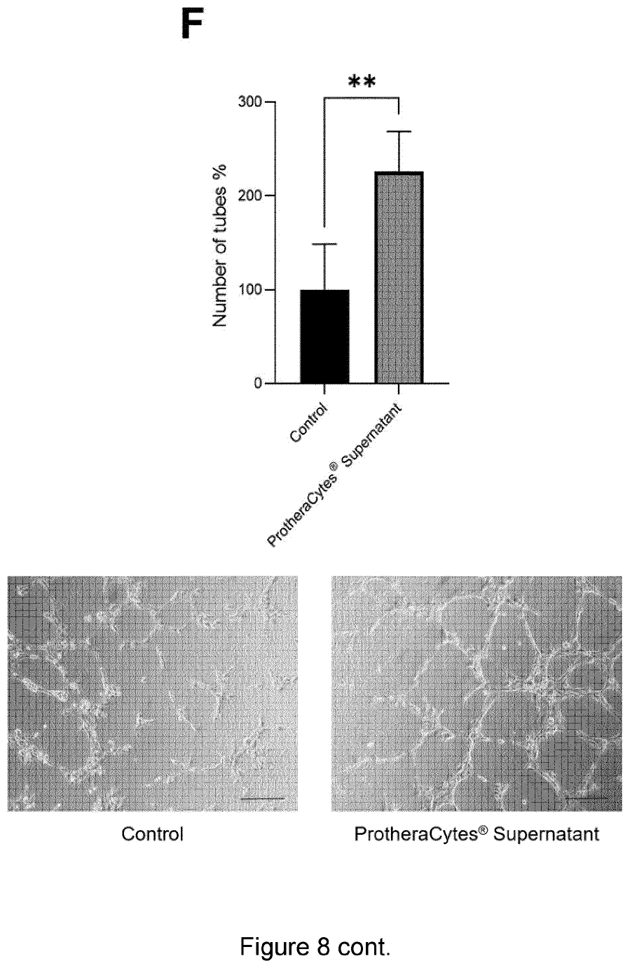

Human CD34+ cells have been shown to secrete VEGF (Bautz et al., 2000) and we wanted to determine the level of VEGF secretion by CD34+ cells after expansion of 16 ProtheraCytes® batches manufactured from AMI patients from the EXCELLENT Phase I/IIb clinicat trial, as well as 4 batches manufactured from healthy donors. The quantification of VEGF concentration by ELISA showed that the culture supernatants of ProtheraCytes® from patients ranged from 185.6 pg/mL to 1032.4 pg/mL with a mean value of 596.2±242.3 pg/mL, and the VEGF concentration from healthy donor cells ranged from 315.3 pg/mL to 718.3 pg/mL with a mean value of 526.2±208.1 pg/mL (FIG. 8A). No significant difference was observed between the VEGF concentrations of patients and healthy donors (FIGS. 8A and B).

Conversely, the concentration of VEGF observed in the StemFeed® culture medium (negative control) ranged from 2.7 pg/mL to 3.0 pg/mL with a mean value of 2.8±0.2 pg/mL, which was significantly lower than the VEGF concentration of patients (p=0.0007) and healthy donors (p=0.087) (FIG. 8B). When the VEGF concentration was normalized by the number of CD34+ cells, the VEGF secreted per cell in AMI patients was 4.4 fg/cell and was not significantly different (p=0.6343) from the VEGF secreted per cell in healthy donors (4.1 fg/cell) (FIG. 8C). Furthermore, the concentration of VEGF in the culture supernatant of the expanded CD34+ cells from AMI patients was significantly correlated with the number of CD34+ cells obtained after expansion (FIGS. 8D and E) (Pearson correlation coefficient r=0.7484; p-value=0.0009).

We then conducted an in vitro tube formation assay with HUVECs to determine if the ProtheraCytes® supernatant containing VEGF has in vitro angiogenic activity. We quantified tube formation in HUVECs that had been cocultured for 6 hours with ProtheraCytes® supernatant or StemFeed® medium as negative control and observed significantly higher tube formation in HUVECs cocultured with ProtheraCytes® supernatant (p=0.0082) (FIG. 8F).

### ProtheraCytes®-Derived Exosome Characterization

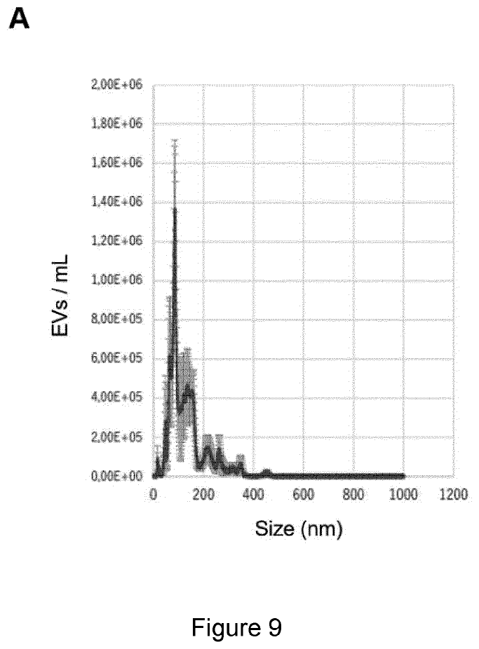

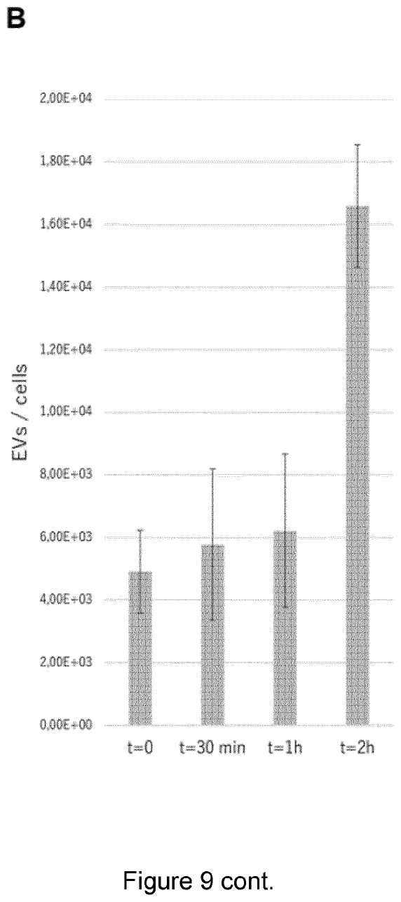

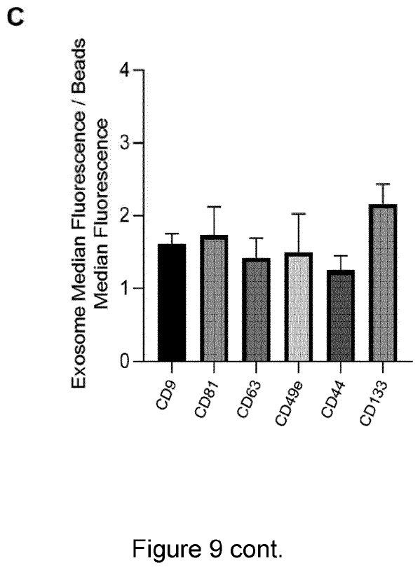

In collaboration with Everzom, ProtheraCytes® were seeded in a bioreactor with controlled mechanical stimulation to collect exosomes. The average size of Protheracytes®-derived exosomes was 86.7±10.17 nm (FIG. 9A). We observed that the number of exosomes secreted by ProtheraCytes® increased over time from around 6,000 extracellular vesicles/cell at 30 minutes to 16,000 extracellular vesicles/cell at 2 hours (FIG. 9B). We also analyzed the marker expression of the exosomes and showed that they express the exosomal markers CD9, CD63, and CD81 as well as the endothelial CD49e, CD44 endothelial, CD133 stem cell markers (FIG. 9C).

miRNA Expression in ProtheraCytes®-Derived Exosomes (CD34Exo)

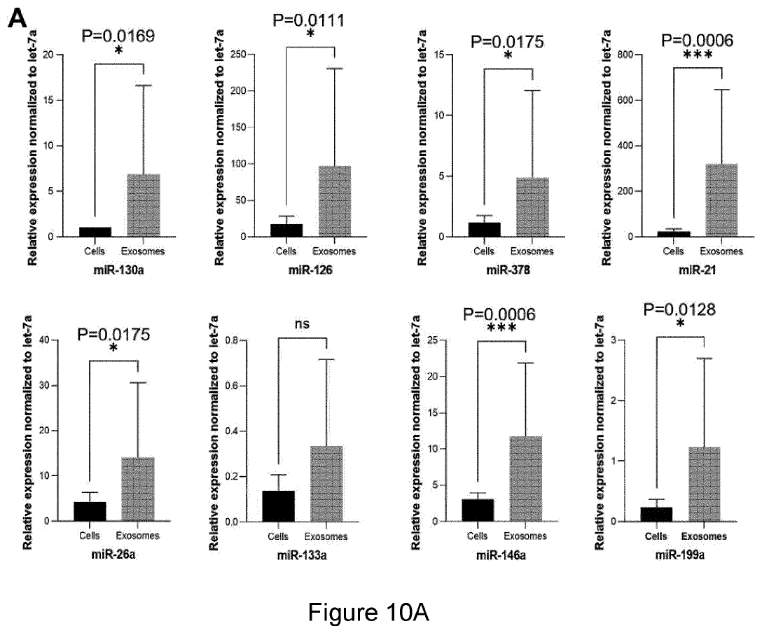

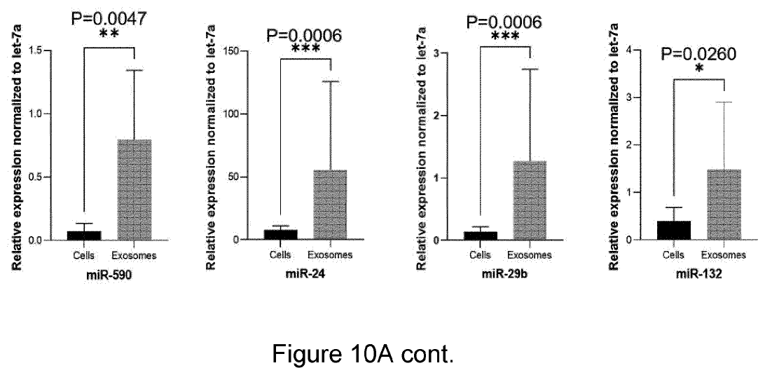

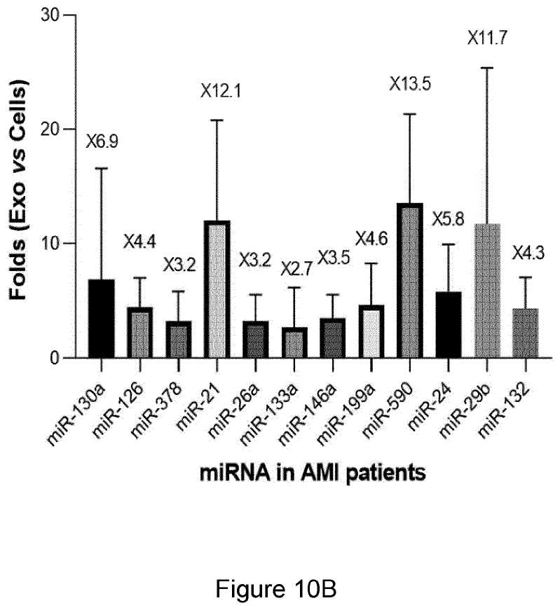

To investigate the proangiogenic activity of ProtheraCytes® derived exosomes (CD34Exo), we first isolated exosomes and characterized them by flow cytometry. We then isolated RNA from CD34Exo and determined the expression levels of miRNAs that have been reported to be key positive regulators of the angiogenesis processes. We assessed the expression levels of miR-126, miR-130a, miR-21, miR-378, and miR-26a, (Sahoo et al., 2011; Mathiyalagan et al., 2017; Templin et al., 2017; Mcneill et al., 2019; Li et al., 2021; Chang et al., 2022) in ProtheraCytes® and their exosomes (CD34Exo). Exosomes were found to be significantly enriched in miR-130a, miR-126, miR-378, miR-26a, and miR-21, compared to ProtheraCytes® (FIG. 10A). The miR-130a and miR-21 were the two most enriched miRNAs in CD34Exo and their expression was 6.9 and 12.1-fold higher, respectively, than in ProtheraCytes® (FIG. 10B). Similarly, we observed significantly higher expression of miR-126 by 4.4-fold, and miR-378 and miR-26a by 3.2-fold in ExoCD34⁺ compared to ProtheraCytes® (FIG. 10B).

These results indicate that ProtheraCytes® are able to secrete exosomes containing proangiogenic miRNAs which might lead to the induction of angiogenesis and contribute to the vascular repair process. We also investigated the expression level of miR-146a, which has been shown to attenuate apoptosis (Huang et al., 2016; Scarlatescu et al., 2021) and found that it was also enriched in ExoCD34⁺ by 3.5-fold compared to ProtheraCytes® (FIG. 10B).

Expression of both miR-199a and miR-590 were detected at low levels but were significantly higher in ExoCD34⁺ compared to ProtheraCytes® (FIG. 10A). Interestingly, miR590 was one of the most enriched miRNAs in ExoCD34+with 13.5-fold higher expression and miR-199a was 4.6 times higher in ExoCD34+ than in cells (FIG. 10B).

Finally, we further examined the expression of miR-133a, miR-24, miR-29b, and miR-132, known to have anti-fibrotic activity (Xiao et al., 2019). As shown in FIGS. 10A and B, miR-29b, miR-24, and miR-132 were significantly enriched in Exo CD34+ and their expression was 11.7, 5.8 and 4.3-fold higher, respectively, than in ProtheraCytes®. On the other hand, there was no significant difference in the expression of miR133 between ExoCD34+ and ProtheraCytes®.

### REFERENCES

Bautz, F., Rafii, S., Kanz, L., and Môhle, R. (2000). Expression and secretion of vascular endothelial growth factor-A by cytokine-stimulated hematopoietic progenitor cells. Possible role in the hematopoietic microenvironment. Exp Hematol 28, 700-706. doi: 10.1016/50301-472x(00)00168-5
Banerjee S, Bentley P, Hamady M, Marley S, Davis J, Shlebak A, Nicholls J, Williamson D A, Jensen S L,
Gordon M, Habib N, Chataway J. Intra-Arterial Immunoselected CD34+ Stem Cells for Acute Ischemic Stroke. Stem Cells Transi Med. 2014 November; 3(11):1322-30. doi: 10.5966/sctm.2013-0178. Epub 2014 Aug
8. PMID: 25107583; PMCID: PMC4214837.
Chen D C, Lin S Z, Fan J R, Lin C H, Lee W, Lin C C, Liu Y J, Tsai C H, Chen J C, Cho D Y, Lee C C, Shyu W C. Intracerebral implantation of autologous peripheral blood stem cells in stroke patients: a randomized phase II study. Cell Transplant. 2014; 23(12):1599-612. doi: 10.3727/096368914X678562. Epub 2014 Jan. 29. PMID: 24480430.
Chang, W.-T., Lin, Y.-W., Huang, P.-S., Lin, Y.-C., Tseng, S.-Y., Chao, T.-H., et al. (2022). Deletion of MicroRNA-21 Impairs Neovascularization Following Limb Ischemia: From Bedside to Bench. Front.
Cardiovasc. Med. 9, 826478. doi: 10.3389/fcvm.2022.826478.
Gleeson J G, Lin P T, Flanagan L A, Walsh C A. Doublecortin is a microtubule-associated protein and is expressed widely by migrating neurons. Neuron. 1999 June;23(2):257-71. doi: 10.1016/s0896-6273(00)80778-3. PMID: 10399933.
Gratama J W, Kraan J, Keeney M et al. Validation of the single-platform ISHAGE method for CD34(+) hematopoietic stem and progenitor cell enumeration in an international multicentre study. Cytotherapy 2003; 5:55-65.
Huang, W., Tian, S.-S., Hang, P.-Z., Sun, C., Guo, J., Du, Z.-M., 2016. Combination of microRNA-21 and microRNA-146a Attenuates Cardiac Dysfunction and Apoptosis During Acute Myocardial Infarction in Mice. Mol. Ther. Nucleic Acids 5, e296.
Li, Y., Chen, X., Jin, R., Chen, L., Dang, M., Cao, H., et al. (2021). Injectable hydrogel with MSNs/microRNA-21-5p delivery enables both immunomodification and enhanced angiogenesis for
   myocardial infarction therapy in pigs. Sci. Adv. 7, eabd6740. doi: 10.1126/sciadv.abd6740.
Mathiyalagan, P., Liang, Y., Kim, D., Misener, S., Thorne, T., Kamide, C. E., et al. (2017). Angiogenic Mechanisms of Human CD34+ Stem Cell Exosomes in the Repair of Ischemic Hindlimb. Circ. Res. 120, 1466-1476. doi: 10.1161/CIRCRESAHA.116.310557
Mcneill, B., Ostojic, A., Rayner, K. J., Ruel, M., and Suuronen, E. J. (2019). Collagen biomaterial stimulates the production of extracellular vesicles containing microRNA-21 and enhances the proangiogenic function of CD34+ cells. FASEB j. 33, 4166-4177. doi: 10.1096/fj.201801332R
Nystedt J, Mákinen S, Lame J, Jolkkonen J. Human cord blood CD34+ cells and behavioral recovery following focal cerebral ischemia in rats. Acta Neurobiol Exp (Wars). 2006; 66(4):293-300. PMID: 17265691.
Sahoo, S., Klychko, E., Thorne, T., Misener, S., Schultz, K. M., Millay, M., et al. (2011). Exosomes From
   Human CD34+ Stem Cells Mediate Their Proangiogenic Paracrine Activity. Circulation Research 109, 724-728. doi: 10.1161/CIRCRESAHA.111.253286.
Saucourt, C., Vogt, S., Merlin, A., Valat, C., Criquet, A., Harmand, L., Birebent, B., Rouard, H., Himmelspach, C., Jeandidier, É., Chartois-Leauté, A.-G., Derenne, S., Koehl, L., Salem, J.-E., Hulot, J.-
   S., Tancredi, C., Aries, A., Judé, S., Martel, E., Richard, S., Douay, L., Hénon, P., 2019. Design and Validation of an Automated Process for the Expansion of Peripheral Blood-Derived CD34+ Cells for Clinicat Use After Myocardial Infarction. Stem Cells Transi. Med. 8, 822-832.
Scárlátescu, A. I., Micheu, M. M., Popa-Fotea, N.-M., and Dorobaniu, M. (2021). MicroRNAs in Acute ST Elevation Myocardial Infarction—A New Tool for Diagnosis and Prognosis: Therapeutic Implications.
International Journal of Molecular Sciences 22, 4799. doi: 10.3390/ijms22094799.
Templin, C., Volkmann, J., Emmert, M. Y., Mocharla, P., Múller, M., Kraenkel, N., et al. (2017). Increased Proangiogenic Activity of Mobilized CD34+Progenitor Cells of Patients With Acute ST-Segment—Elevation Myocardial Infarction: Role of Differential MicroRNA-378 Expression.
Arteriosclerosis, Thrombosis, and Vascular Biology 37, 341-349. doi: 10.1161/ATVBAHA.116.308695.
Wang, J., and Martin, J. F. (2014). Macro advances in microRNAs and myocardial regeneration. Curr Opin Cardiol 29, 207-213. doi: 10.1097/HCO.0000000000000050.
Wang L, Ji H, Li M, Zhou J, Bai W, Zhong Z, Li N, Zhu D, Zhang Z, Liu Y, Wu M. Intrathecal Administration of Autologous CD34 Positive Cells in Patients with Past Cerebral Infarction: A Safety Study. ISRN Neurol. 2013 Sep. 25; 2013:128591. doi: 10.1155/2013/128591. PMID: 24187628; PMCID: PMC3800652.

1. A method of treating stroke comprising intranasally administering a population of CD34+ to a subject.

2. (canceled)

3. The method of claim 1, wherein the CD34+ cells are isolated from umbilical cord blood or whole blood.

4. The method of claim 1, wherein the CD34+ cells are administered to the subject within 1 day, 2 days, 3 days, or within 4 days, of the stroke event.

5. The method of claim 1, wherein the method comprises administering at least 7×10⁶ CD34+ cells.

6. The method of claim 1, wherein the CD34+ cells express one or more paracrine factors, and/or VEGF.

7. The method of claim 1, wherein the amount of VEGF expressed by the CD34+ cells into a culture medium is at least about 150 pg/ml, or wherein the amount of VEGF expressed by the CD34+ cells into a culture medium is at least about 4 fg/cell.

8. The method of claim 1, wherein the CD34+ cells express one or more or each of miR126, miR130a, miR21, miR26a, miR378a, miR146a, miR21, miR199a, miR590, miR133a, miR-24, miR29b, and miR132.

9. The method of claim 1, wherein the CD34+ cell viability is at least about 90%, and/or wherein the CD34+ cell purity is at least about 75%.

10. The method of claim 1, wherein the CD34+ cells are a cultured and/or expanded population, and/or purified, and/or, wherein the CD34+ cells are an isolated population.

11. The method of claim 1, wherein the population comprises at least
(i) about 75% CD34+ cells, and/or
(ii) about 15% monocytes or lower, and/or
(iii) about 5% granulocytes or lower, and/or
(iv) about 3% lymphocytes or lower.

12. The method of claim 1, wherein the number of cells administered per dose is about 7×10⁶.

13. The method of claim 1, wherein the volume of cells administered per nostril is about 1 ml and/or the total volume administered per dose is about 2 ml.

14. The method of claim 1, wherein the CD34+ cells are provided as a sterile suspension, and/or wherein the population is provided in the form of a pharmaceutical composition, optionally comprising buffer.

15. The method of claim 1, wherein the CD34+ cells increase the expression of doublecortin DCX and/or VEGFR1.

16. A method of treating stroke comprising administering a population of CD34+ cells to a subject:
(a) wherein the CD34+ cells are administered to the subject intranasally, wherein the CD34+ cells secrete paracrine factors in an area affected by stroke thereby treating stroke; and/or
(b) wherein the CD34+ cells are administered to the subject intranasally, wherein the CD34+ cells promote angiogenesis in an area affected by stroke, thereby treating stroke; and/or
(c) wherein the CD34+ cells are administered to the subject intranasally, and wherein the CD34+ cells promote neurogenesis; and/or
(d) wherein the CD34+ cells are administered to the subject intranasally, and wherein the CD34+ cells promote reduction in infarct size, and/or reduce cell loss in the infarct area and/or peri-infarct area and/or inflammation in the infarct area and/or peri-infarct area, thereby treating stroke; and/or
(e) wherein the CD34+ cells are administered to the subject intranasally, and wherein the CD34+ cells promote motor and/or neurological recovery, thereby treating stroke.

17. A method of stimulating angiogenesis or neurogenesis comprising administering a population of CD34+ cells to a subject.
